(19) United States
(12) Patent Application Publication
(10) Pub. No.: US 2012/0277169 A1
(43) Pub. Date: Nov. 1, 2012

(54) NYASOL AND ANALOGS THEREOF FOR THE TREATMENT OF ESTROGEN RECEPTOR BETA-MEDIATED DISEASES

(75) Inventor: Isaac Cohen, Piedmont, CA (US)

(73) Assignee: BioNovo, Inc., Emeryville, CA (US)

(21) Appl. No.: 13/286,780

(22) Filed: Nov. 1, 2011

Related U.S. Application Data
(62) Division of application No. 12/484,067, filed on Jun. 12, 2009.
(60) Provisional application No. 61/061,494, filed on Jun. 13, 2008.

(51) Int. Cl. A61K 31/05; A61K 31/255; A61K 31/7034; A61P 5/24; A61P 19/10; A61P 35/00; A61P 9/00; A61P 25/00; A61P 15/02; A61P 15/12; A61P 13/00; A61P 25/24; C07C 37/14; C12N 5/071; A61K 31/085

(52) U.S. Cl. 514/23; 514/733; 514/720; 514/517; 568/729; 435/375

(57) ABSTRACT

Estrogenic compositions comprising nyasol and analogs thereof are provided. Also provided are methods of using said extracts to achieve an estrogenic effect, especially in a human, e.g. a female human. In some embodiments, the methods include treatment of climacteric symptoms. In some embodiments, the methods include treatment of estrogen receptor positive cancer, such as estrogen responsive breast cancer. In some embodiments, the methods include treatment or prevention of osteoporosis.

### CROSS-REFERENCE

This application is a division of U.S. patent application Ser. No. 12/484,067, filed Jun. 12, 2009, which is a non-provisional application claiming priority to U.S. Provisional Application No. 61/061,494, filed Jun. 13, 2008, which are incorporated herein by reference in their entirety.

### FIELD OF THE INVENTION

The present invention relates to methods of using Nyasol and analogs thereof for the preparation of medicaments for the treatment of estrogen receptor beta- (ERβ-) mediated conditions. The invention further relates to methods of using Nyasol and analogs thereof for the treatment of ERβ-mediated conditions.

### BACKGROUND OF THE INVENTION

Hormone replacement therapy (HRT) has been used successfully to treat a variety of conditions, such as osteoporosis, increased risk of cardiovascular disease in post-menopausal women and climacteric symptoms, such as hot flashes, decreased libido and depression. However, HRT with estradiol (E₂), either alone or in combination with progestin, can lead to undesirable effects. In fact, a recent Women's Health Initiative (WHI) study was abruptly halted when preliminary results showed that HRT was associated with a 35% increased risk of breast cancer.

Breast cancer can be treated or prevented by using a so-called selective estrogen receptor modulator (SERM), such as tamoxifen. (Before the approval of tamoxifen, breast cancer treatment of pre-menopausal women often included removing the ovaries in order to reduce the cancer-stimulating effect of estrogen.) Tamoxifen appears to selectively block the cancer-inducing effects of estrogen in breast tissues of pre-menopausal women. Another SERM, raloxifene, has been approved for treatment of osteoporosis as an alternative to estrogen replacement. In addition to selectively inducing estrogenic effects in bone tissue, long-term administration of raloxifene was also shown to be associated with reduction in the rate of breast cancer in the Multiple Outcomes of Raloxifene Evaluation (MORE) study.

While SERMs such as tamoxifen and raloxifene provide selective reduction in estrogen's cancer-inducing effects in the breast, they are not without their risks. For example both tamoxifen and raloxifene therapy have been associated with increased incidence of hot flushes, and tamoxifen therapy has been shown to increase the risk of uterine (endometrial) cancer.

Despite the success of estrogen replacement therapy in treating osteoporosis, coronary heart disease and climacteric symptoms, and of SERMs like tamoxifen and raloxifene in treating breast cancer and osteoporosis, there remains a need for compositions having estrogenic properties. Additionally, given the increasing cost of producing drug compounds, there is a need for additional estrogenic compositions that may be obtained from natural sources.

### SUMMARY OF THE INVENTION

The present inventor has identified a need for estrogenic compositions useful for the treatment of one or more disease states associated with the estrogen receptor. The inventor has also identified a need for estrogenic compositions that do not increase the risk or likelihood that a patient administered the compositions will suffer from another disease state associated with an estrogen receptor. The inventor has likewise recognized a need for an estrogenic composition that will reduce the risk of one or more estrogen receptor mediated disease states while, at the same time, treating another estrogen receptor mediated disease state. The inventor has also identified a need for estrogenic compositions that are readily obtained from natural sources, as well as a need for methods of making and using such estrogenic compositions. The disclosure herein meets such needs and provides related advantages as well.

Thus, embodiments described herein provide a pharmaceutical composition, comprising an amount of at least one isolated and purified member of the group consisting of compounds (a), (b), (c), (d), (e), (f), (g) and (h), wherein the amount is sufficient to modulate estrogen receptor beta (ERβ) in a multicellular organism:

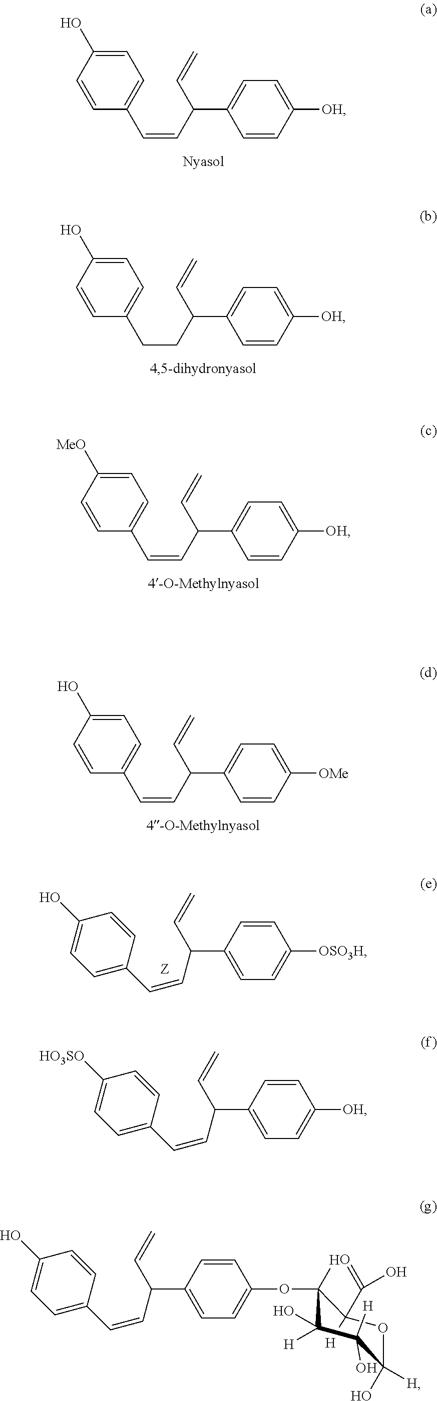

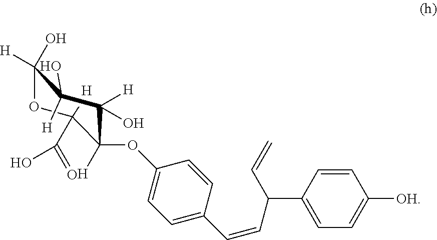

Hereinafter, compounds (a), (b), (c), (d), (e), (f), (g) and (h) may be referred to simply as (a), (b), (c), (d), (e), (f), (g) and (h), a usage which will be clear in context.

In some embodiments, the composition comprises two or more, three or more or all four of (a), (b), (c), (d), (e), (f), (g) and (h). Some embodiments provide the use of such composition for the manufacture of a medicament. In particular, a composition or medicament described herein possesses an estrogen receptor beta-agonistic effect. In some embodiments, the composition or medicament possesses a selective estrogen receptor beta-agonistic effect. In some embodiments, the composition or medicament antagonizes estrogen receptor alpha or has little or no measurable effect on estrogen receptor alpha. In some embodiments, the estrogenic effect is at least one effect selected from the group consisting of: treating or preventing at least one climacteric symptom; treating or preventing osteoporosis; treating or preventing uterine cancer; and treating or preventing cardiovascular disease. In some embodiments, the estrogenic effect includes treating or preventing at least one climacteric symptom selected from the group consisting of treating or preventing hot flashes, insomnia, vaginal dryness, decreased libido, urinary incontinence and depression. In some embodiments, the estrogenic effect includes treating or preventing osteoporosis. In some embodiments, the estrogenic effect includes treating or preventing hot flashes. In some embodiments, the estrogenic effect includes treating or preventing uterine cancer or breast cancer. In some embodiments, the estrogenic effect does not include increasing the risk of mammary hyperplasia, mammary tumor, uterine hyperplasia, uterine tumor, cervical hyperplasia, cervical tumor, ovarian hyperplasia, ovarian tumor, fallopian tube hyperplasia, fallopian tube tumor. In some embodiments, the estrogenic effect includes decreasing the risk of mammary hyperplasia, mammary tumor, uterine hyperplasia, uterine tumor, cervical hyperplasia, cervical tumor, ovarian hyperplasia, ovarian tumor, fallopian tube hyperplasia, fallopian tube tumor. Some embodiments provide for the use of a composition of a composition described herein for the preparation of a medicament.

Some embodiments described herein provide a method of eliciting an estrogenic effect, comprising administering to a subject an estrogenically effective amount of one comprising an amount of at least one isolated and purified member of the group consisting of compounds (a), (b), (c), (d), (e), (f), (g) and (h), wherein the amount is sufficient to modulate estrogen receptor beta (ERβ) in a multicellular organism:

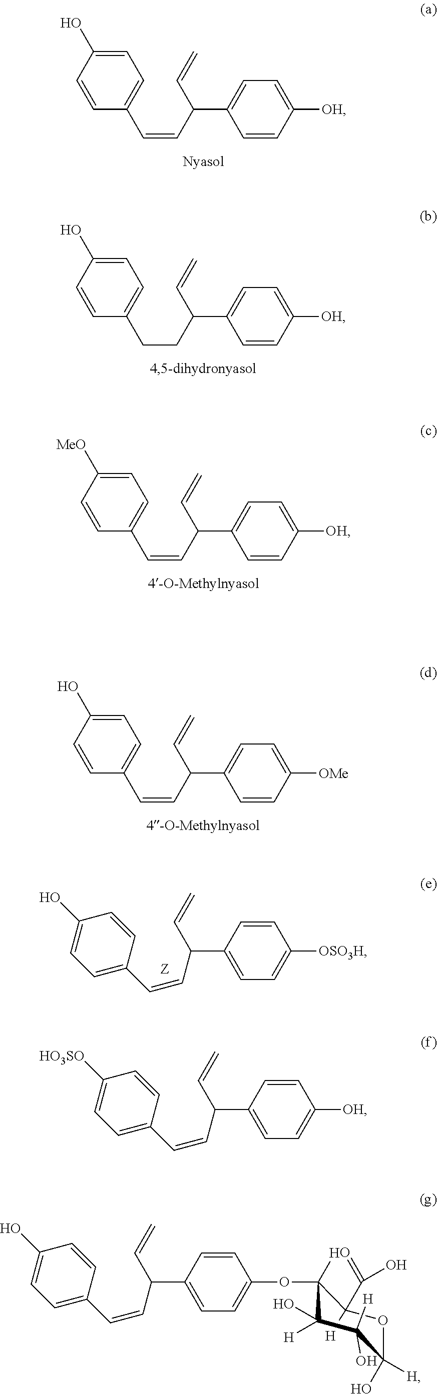

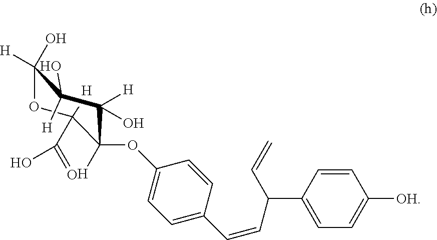

In some embodiments, the composition comprises two or more, three or more or all four of (a), (b), (c), (d), (e), (f), (g) and (h). Some embodiments provide the use of such composition for the manufacture of a medicament. In particular, a composition or medicament described herein possesses an estrogen receptor beta-agonistic effect. In some embodiments, the composition or medicament possesses a selective estrogen receptor beta-agonistic effect. In some embodiments, the composition or medicament antagonizes estrogen receptor alpha or has little or no measurable effect on estrogen receptor alpha. In some embodiments, the estrogenic effect is at least one effect selected from the group consisting of: treating or preventing at least one climacteric symptom; treating or preventing osteoporosis; treating or preventing uterine cancer; and treating or preventing cardiovascular disease. In some embodiments, the estrogenic effect includes treating or preventing at least one climacteric symptom selected from the group consisting of treating or preventing hot flashes, insomnia, vaginal dryness, decreased libido, urinary incontinence and depression. In some embodiments, the estrogenic effect includes treating or preventing osteoporosis. In some embodiments, the estrogenic effect includes treating or preventing hot flashes. In some embodiments, the estrogenic effect includes treating or preventing uterine cancer or breast cancer. In some embodiments, the estrogenic effect does not include increasing the risk of mammary hyperplasia, mammary tumor, uterine hyperplasia, uterine tumor, cervical hyperplasia, cervical tumor, ovarian hyperplasia, ovarian tumor, fallopian tube hyperplasia, fallopian tube tumor. In some embodiments, the estrogenic effect includes decreasing the risk of mammary hyperplasia, mammary tumor, uterine hyperplasia, uterine tumor, cervical hyperplasia, cervical tumor, ovarian hyperplasia, ovarian tumor, fallopian tube hyperplasia, fallopian tube tumor. Some embodiments provide for the use of a composition of a composition described herein for the preparation of a medicament Some embodiments described herein provide a method of activating a gene under control of an estrogen response element, comprising administering to a cell having an estrogen response element operatively linked to the gene and an estrogen receptor an amount of a composition of described herein sufficient to activate said gene. In some embodiments, said cell is in vitro. In some embodiments, said cell is in vivo. In some embodiments, said cell is in an ERα+ breast tissue. In some embodiments, said cell is in an ERβ+ breast tissue. In some embodiments, said cell is in an ERα/ERβ+ breast tissue. In some embodiments, said estrogen response element is expressed in a transformed cell. In some embodiments, the estrogen response element and the estrogen receptor are expressed in a transformed cell. In some embodiments, said estrogen response element is heterologously expressed in the cell. In some embodiments, the estrogen response element and the estrogen receptor are heterologously expressed in the cell. In some embodiments, cell is selected from the group consisting of a U937, a U2OS, a MDA-MB-435 and a MCF-7 cell transformed with an ERE-controlled gene. In some embodiments, the cell expresses ERα. In some embodiments, the cell expresses ERβ. In some embodiments, ERE-controlled gene is ERE-tk-Luc.

Some embodiments described herein provide a method of repressing expression of a TNF RE-controlled gene, comprising administering to a cell comprising a gene under control of a TNF response element and an estrogen receptor an amount of a composition described herein effective to repress said TNF RE-controlled gene. In some embodiments, the TNF RE-controlled gene is TNF-α. In some embodiments, the TNF RE-controlled gene is TNF RE-Luc. In some embodiments, said cell is in vitro. In some embodiments, said cell is in vivo. In some embodiments, said cell is in an ER+ breast tissue. In some embodiments, said cell is in an ERα+ breast tissue. In some embodiments, said cell is in an ERβ+ breast tissue. In some embodiments, said TNF response element is endogenously expressed in the cell. In some embodiments, both the TNF response element and the estrogen receptor are endogenously expressed in the cell. In some embodiments, said TNF response element is heterologously expressed in the cell. In some embodiments, the TNF response element and the estrogen receptor are heterologously expressed in the cell. In some embodiments, said cell contains an estrogen receptor gene, is transformed with a TNF response element-controlled gene, and is selected from the group consisting of a U937, a U2OS, a MDA-MB-435 and a MCF-7 cell. In some embodiments, the estrogen receptor gene is a gene expressing ERα. In some embodiments, the estrogen receptor gene is a gene expressing ERβ.

Some embodiments described herein provide a method of preparing nyasol, comprising:

(a) protecting the hydroxy group of 4-iodophenol with MOMCl to form a protected intermediate:

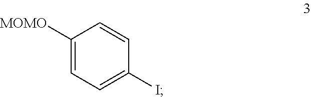

(b) contacting 3 with trimethylsilylacetylene in the presence of bis[triphenylphosphine]palladium dichloride and CuI in diethylamine to form:

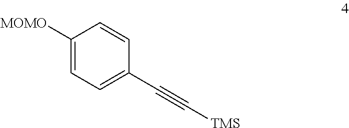

(c) reacting 4-hydroxybenzadehyde with MOMCl to form:

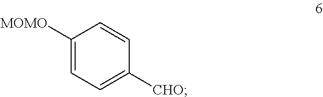

(d) reacting 6 with ethynlymagneciumbromide to form:

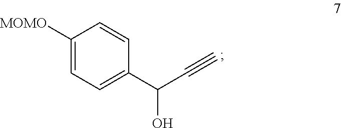

(e) reacting 4 and 7 to form 8:

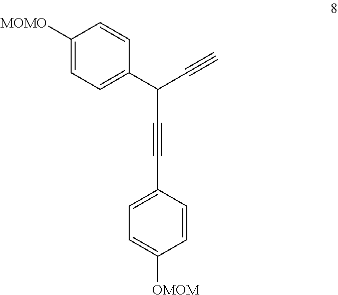

(f) reducing the triple bonds in 8 to form 9:

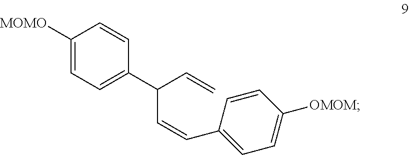

and
(g) removing the MOM protective groups to form 1 (nyasol):

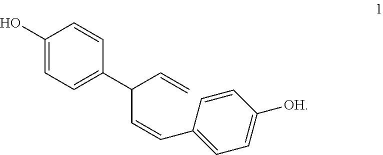

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a graph of luciferase expression in U937 (human monocytes) cells transformed with DNA encoding estrogen response element linked to the minimal thymidine kinase (tk) promoter and a sequence encoding luciferase (Luc) in response to varying concentrations of estradiol (E₂) in the presence of either estrogen receptor alpha (ERα), estrogen receptor beta (ERβ) or both. ERβ has much less stimulatory effect on the ERE than does ERα in the presence of E₂.

FIG. 2 is a graph of luciferase expression in MDA-MB-435 (human metastatic breast cancer) cells transformed with DNA encoding estrogen response element linked to the minimal thymidine kinase (tk) promoter and a sequence encoding luciferase (Luc) in response to varying concentrations of estradiol (E₂) in the presence of either estrogen receptor alpha (ERα), estrogen receptor beta (ERβ) or both. ERβ has much less stimulatory effect on the ERE than does ERα in the presence of E₂. Remarkably, when ERα and ERβ are coexpressed in this cell line, ERβ expression greatly reduces the ERE stimulatory effect of ERα in the presence of E₂.

FIG. 3 is a graph comparing luciferase expression in cells transformed with DNA encoding estrogen response element alpha linked to the minimal thymidine kinase (tk) promoter and a sequence encoding luciferase (Luc) in response to varying concentrations of nyasol in the presence of either estrogen receptor alpha (ERα) or estrogen receptor beta (ERβ). The enhanced expression of luciferase in the presence of ERβ versus ERα demonstrates that nyasol is a selective estrogen receptor beta agonist.

FIG. 4 shows the ERβ-selective repression of TNF-ERE.

FIG. 5 compares luciferase expression in cells transformed with DNA encoding estrogen response element alpha linked to the minimal thymidine kinase (tk) promoter and a sequence encoding luciferase (Luc) in response to Nyasol+EtOH, Nyasol+raloxifene, Nyasol+tamoxifen and Nyasol+estradiol (E₂) in the presence of estrogen receptor beta (ERβ).

FIG. 6 shows concentration binding curves for Nyasol with ERβ and ERα.

FIG. 7 shows a comparison of the effects of estradiol (E₂), Nyasol and control (carrier) on kidney capsule xenografts of MCF-7 breast cancer cells. MCF-7 xenografts were introduced into nude mouse kidneys. Mice were randomized to three treatment groups. The estradiol group received 0.5 mg/h E₂ in saline; the Nyasol group received 2.5 mg/h of Nyasol in saline; the control group received saline only. Each treatment group was treated for 28 days, after which mice were euthanized and the kidneys containing the xenografts were excised, photographed and weighed. As can be seen, estradiol agonizes tumor xenograft growth as compared to control, whereas Nyasol inhibits the growth of MCF-7 breast cancer xenografts.

FIG. 8 shows a comparison of the effects of E₂, Nyasol and a control on in vivo uterine weight. Female nude mice were treated with either E₂, Mice were randomized to three treatment groups. The estradiol group received 0.5 mg/h E₂ in saline; the Nyasol group received 2.5 mg/h of Nyasol in saline; the control group received saline only. After 28 days, each mouse was euthanized and its uterus was removed and weighed. As can be seen, E₂ agonizes uterus growth, while Nyasol has the opposite effect, relative to control.

### DETAILED DESCRIPTION OF THE INVENTION

Embodiments disclosed herein provide a pharmaceutical composition, comprising an amount of at least one isolated and purified member of the group consisting of compounds (a), (b), (c), (d), (e), (f), (g) and (h), wherein the amount is sufficient to modulate estrogen receptor beta (ERβ) in a multicellular organism:

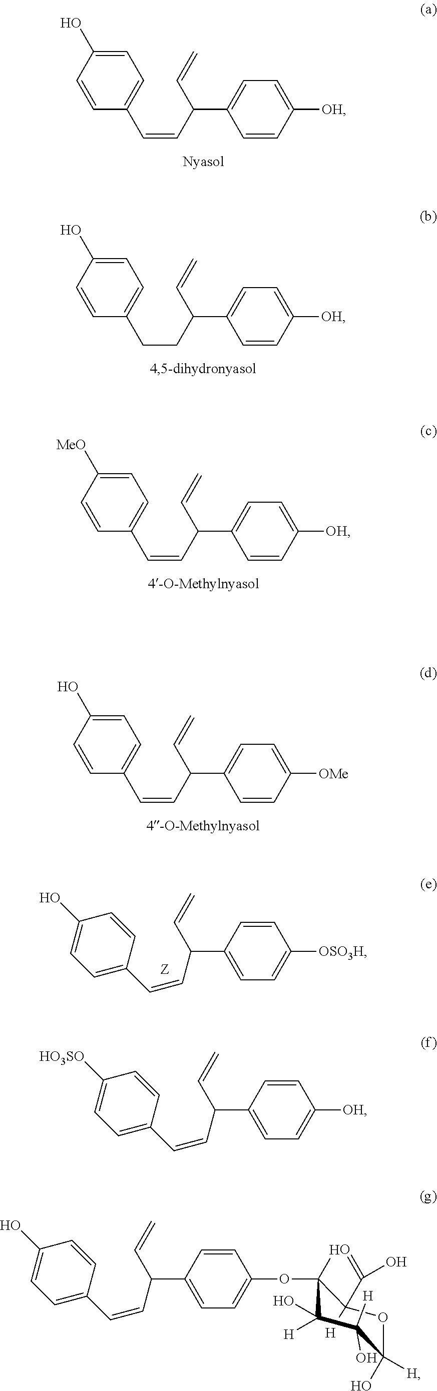

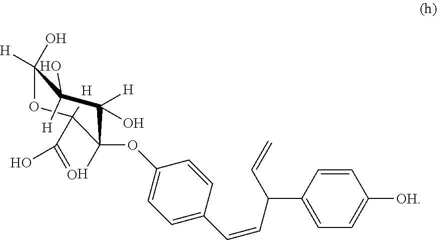

In some embodiments, the composition comprises two or more, three or more or all four of (a), (b), (c), (d), (e), (f), (g) and (h). Some embodiments provide the use of such composition for the manufacture of a medicament. In particular, a composition or medicament described herein possesses an estrogen receptor beta-agonistic effect. In some embodiments, the composition or medicament possesses a selective estrogen receptor beta-agonistic effect. In some embodiments, the composition or medicament antagonizes estrogen receptor alpha or has little or no measurable effect on estrogen receptor alpha. In some embodiments, the estrogenic effect is at least one effect selected from the group consisting of: treating or preventing at least one climacteric symptom; treating or preventing osteoporosis; treating or preventing uterine cancer; and treating or preventing cardiovascular disease. In some embodiments, the estrogenic effect includes treating or preventing at least one climacteric symptom selected from the group consisting of treating or preventing hot flashes, insomnia, vaginal dryness, decreased libido, urinary incontinence and depression. In some embodiments, the estrogenic effect includes treating or preventing osteoporosis. In some embodiments, the estrogenic effect includes treating or preventing hot flashes. In some embodiments, the estrogenic effect includes treating or preventing uterine cancer or breast cancer. In some embodiments, the estrogenic effect does not include increasing the risk of mammary hyperplasia, mammary tumor, uterine hyperplasia, uterine tumor, cervical hyperplasia, cervical tumor, ovarian hyperplasia, ovarian tumor, fallopian tube hyperplasia, fallopian tube tumor. In some embodiments, the estrogenic effect includes decreasing the risk of mammary hyperplasia, mammary tumor, uterine hyperplasia, uterine tumor, cervical hyperplasia, cervical tumor, ovarian hyperplasia, ovarian tumor, fallopian tube hyperplasia, fallopian tube tumor. Some embodiments provide for the use of a composition of a composition described herein for the preparation of a medicament.

Some embodiments described herein provide a method of eliciting an estrogenic effect, comprising administering to a subject an estrogenically effective amount of one comprising an amount of at least one isolated and purified member of the group consisting of compounds (a), (b), (c), (d), (e), (f), (g) and (h), wherein the amount is sufficient to modulate estrogen receptor beta (ERβ) in a multicellular organism:

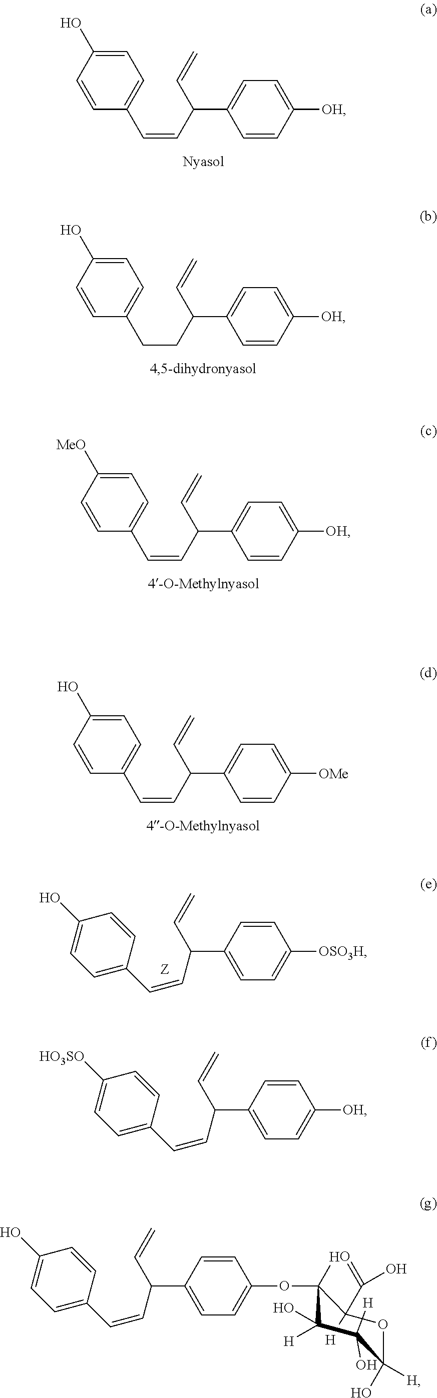

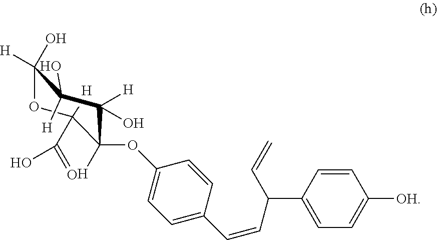

In some embodiments, the composition comprises two or more, three or more or all four of (a), (b), (c), (d), (e), (f), (g) and (h). Some embodiments provide the use of such composition for the manufacture of a medicament. In particular, a composition or medicament described herein possesses an estrogen receptor beta-agonistic effect. In some embodiments, the composition or medicament possesses a selective estrogen receptor beta-agonistic effect. In some embodiments, the composition or medicament antagonizes estrogen receptor alpha or has little or no measurable effect on estrogen receptor alpha. In some embodiments, the estrogenic effect is at least one effect selected from the group consisting of: treating or preventing at least one climacteric symptom; treating or preventing osteoporosis; treating or preventing uterine cancer; and treating or preventing cardiovascular disease. In some embodiments, the estrogenic effect includes treating or preventing at least one climacteric symptom selected from the group consisting of treating or preventing hot flashes, insomnia, vaginal dryness, decreased libido, urinary incontinence and depression. In some embodiments, the estrogenic effect includes treating or preventing osteoporosis. In some embodiments, the estrogenic effect includes treating or preventing hot flashes. In some embodiments, the estrogenic effect includes treating or preventing uterine cancer or breast cancer. In some embodiments, the estrogenic effect does not include increasing the risk of mammary hyperplasia, mammary tumor, uterine hyperplasia, uterine tumor, cervical hyperplasia, cervical tumor, ovarian hyperplasia, ovarian tumor, fallopian tube hyperplasia, fallopian tube tumor. In some embodiments, the estrogenic effect includes decreasing the risk of mammary hyperplasia, mammary tumor, uterine hyperplasia, uterine tumor, cervical hyperplasia, cervical tumor, ovarian hyperplasia, ovarian tumor, fallopian tube hyperplasia, fallopian tube tumor. Some embodiments provide for the use of a composition of a composition described herein for the preparation of a medicament

Some embodiments described herein provide a method of activating a gene under control of an estrogen response element, comprising administering to a cell having an estrogen response element operatively linked to the gene and an estrogen receptor an amount of a composition of described herein sufficient to activate said gene. In some embodiments, said cell is in vitro. In some embodiments, said cell is in vivo. In some embodiments, said cell is in an ERα+ breast tissue. In some embodiments, said cell is in an ERβ+ breast tissue. In some embodiments, said cell is in an ERα/ERβ+ breast tissue. In some embodiments, said estrogen response element is expressed in a transformed cell. In some embodiments, the estrogen response element and the estrogen receptor are expressed in a transformed cell. In some embodiments, said estrogen response element is heterologously expressed in the cell. In some embodiments, the estrogen response element and the estrogen receptor are heterologously expressed in the cell. In some embodiments, cell is selected from the group consisting of a U937, a U2OS, a MDA-MB-435 and a MCF-7 cell transformed with an ERE-controlled gene. In some embodiments, the cell expresses ERα. In some embodiments, the cell expresses ERβ. In some embodiments, ERE-controlled gene is ERE-tk-Luc.

Some embodiments described herein provide a method of repressing expression of a TNF RE-controlled gene, comprising administering to a cell comprising a gene under control of a TNF response element and an estrogen receptor an amount of a composition described herein effective to repress said TNF RE-controlled gene. In some embodiments, the TNF RE-controlled gene is TNF-α. In some embodiments, the TNF RE-controlled gene is TNF RE-Luc. In some embodiments, said cell is in vitro. In some embodiments, said cell is in vivo. In some embodiments, said cell is in an ER+ breast tissue. In some embodiments, said cell is in an ERα+ breast tissue. In some embodiments, said cell is in an ERβ+ breast tissue. In some embodiments, said TNF response element is endogenously expressed in the cell. In some embodiments, both the TNF response element and the estrogen receptor are endogenously expressed in the cell. In some embodiments, said TNF response element is heterologously expressed in the cell. In some embodiments, the TNF response element and the estrogen receptor are heterologously expressed in the cell. In some embodiments, said cell contains an estrogen receptor gene, is transformed with a TNF response element-controlled gene, and is selected from the group consisting of a U937, a U2OS, a MDA-MB-435 and a MCF-7 cell. In some embodiments, the estrogen receptor gene is a gene expressing ERα. In some embodiments, the estrogen receptor gene is a gene expressing ERβ.

Some embodiments described herein provide a method of preparing nyasol, comprising:

(a) protecting the hydroxy group of 4-iodophenol with MOMCl to form a protected intermediate:

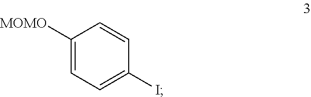

(b) contacting 3 with trimethylsilylacetylene in the presence of bis[triphenylphosphine]palladium dichloride and CuI in diethylamine to form:

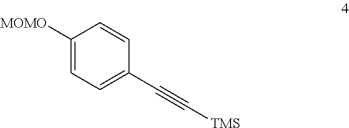

(c) reacting 4-hydroxybenzadehyde with MOMCl to form:

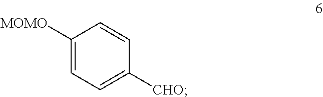

(d) reacting 6 with ethynlymagneciumbromide to form:

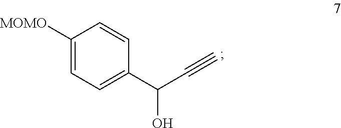

(e) reacting 4 and 7 to form 8:

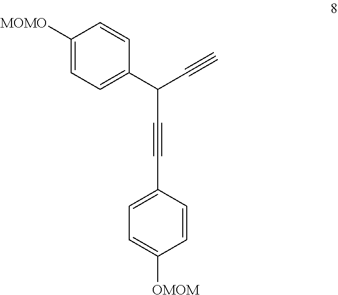

(f) reducing the triple bonds in 8 to form 9:

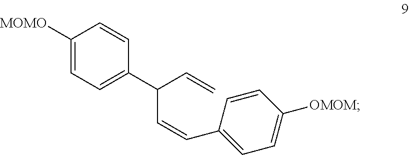

and
(g) removing the MOM protective groups to form 1 (nyasol):

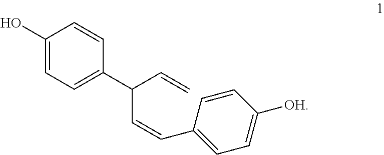

Breast neoplasms are the most common cancers diagnosed in women. In 2000, 184,000 new cases of breast cancer were diagnosed and 45,000 women died from breast cancer. Although the cause of breast cancer is probably multifactorial, there is compelling clinical, epidemiological and biological research that indicate estrogens promote breast cancer: (a) Hormone replacement therapy (HRT) is associated with a 35% increased risk of breast cancer by a meta-analysis of 51 studies; (b) Breast cancer can be prevented with tamoxifen or raloxifene, which bind to ERs and antagonize the actions of estrogens in breast cells; (c) Bilateral oophorectomy in premenopausal women with breast cancer leads to increased survival; (d) Greater exposure to estrogens (early menarche or late menopause, relative risk=1.3 and 1.5 to 2.0, respectively) increases the incidence of breast cancer; (e) Estrogens increase the proliferation of ER positive breast cancer cells; and (f) Estrogens increase the production of growth promoting genes, such as cyclin Dl, c-myc, and c-fos.

Approximately 60-70% of breast tumors contain estrogen receptors. For several decades, breast tumors have been analyzed for the presence of ERs. Approximately 70% of ER+ tumors are responsive to antiestrogen therapy. This observation has led to the notion that ER+ tumors have a better prognosis than ER negative tumors. However, the discovery of ERβ has complicated these interpretations and has raised some profound clinical questions. Understanding the role of ERα and ERβ is of paramount importance, because the current methods of determining whether tumors are ER+ uses an antibody that only detects ERα. Thus, most studies examining the effects ERs in breast tumors on clinical outcomes reflect the ERα status only. However, several recent studies have detected the presence of ERβ mRNA in human breast tumors. Most of the studies relied on RT-PCR to measure ERβ, because of the lack of specific and sensitive antibodies to ERβ. Dotzlaw et al. were the first to detect ERβ in breast tumor biopsies by RT-PCR. They found 70% of the breast tumors expressed ERβ and 90% expressed ERα. Furthermore, they demonstrated that several ER negative cell lines also express ERβ mRNA. These findings suggest that ERβ is highly expressed in breast tumors, and that both ERα and ERβ are often coexpressed in many tumors. In fact, some ER− tumors contain ERβ. Dotzlaw et al. also showed that ERβ mRNA is significantly lower in ER+/PR− (PR being progestin receptor) tumors compared to ER+/PR+ tumors. The authors suggested that this observation indicates that ERβ expression is associated with a poorer prognosis, because ER+/PR+ are more likely to respond to tamoxifen. Other studies suggest that the presence of ERβ confers a poor prognosis. Speirs et al. found that most breast tumors express ERβ mRNA alone or in combination with ERα mRNA. Those tumors that express both ERα and ERβ mRNA were associated with positive lymph nodes and tended to be characterized as higher grade tumors. Furthermore, increased ERβ expression occurs in MCF-10F cells treated with chemical carcinogens, suggesting that the expression of ERβ may contribute to the initiation and progression of breast cancer. Recently, Jensen et al. analyzed the expression of ERβ in 29 invasive breast tumors by immunohistochemistry (IHC). They found that ERβ expression was associated with an elevation of specific markers of cell proliferation, Ki67 and cyclin A. Moreover, the highest expression of these proliferation markers was present in ERα+/ERβ+ tumors. Although the number of ERα−/ERβ+ cases were very small (n=7) the authors suggested that ERβ mediates cell proliferation in breast tumors. Speirs et al. also reported ERβ mRNA is significantly elevated in the tamoxifen-resistant tumors compared to tamoxifen-sensitive tumors.

In contrast, other studies indicate that the presence of ERβ confers a favorable prognosis. Iwao et al. demonstrated that ERα mRNA is up-regulated and ERβ mRNA is down-regulated as breast tumors progress from preinvasive to invasive tumors. Using IHC of frozen tumor sections Jarvinen et al. found that ERβ expression was associated with negative axillary node status, low grade, and low S-phase fraction. A study by Omoto et al. also found that ERβ positive tumors correlated with a better prognosis than ERβ negative tumors, because the disease-free survival rate was higher in tumors containing ERβ. ERβ expression also showed a strong association with the presence of progesterone receptors and well-differentiated breast tumors. It has also been reported that the levels of ERβ are highest in normal mammary tissue and that it decreases as tumors progress from pre-cancerous to cancerous lesions. These studies indicate that ERβ may function as a tumor suppressor and that the loss of ERβ promotes breast carcinogenesis. In a study by Mann et al. it was shown that the expression of ERβ in more than 10% of cancer cells was associated with better survival in women treated with tamoxifen. The aggregate of these studies indicates the presence of ERβ confers a favorable prognosis. Consistent with RT-PCR and IHC data is a report that showed that adenovirus-mediated expression of ERβ resulted in a ligand-independent inhibition of proliferation of the ER negative cell line, MDA-MB-231.

These results demonstrate that the role of ERβ in the pathogenesis and prognosis of breast cancer is unclear. Several reasons may explain the apparent discrepancy among these studies. First, there may be a poor correlation between ERβ mRNA and ERβ protein. This notion is consistent with the presence of ERβ mRNA in some ER negative cell lines that do not have detectable ERs by ligand binding assays. Second, the IHC studies used different commercially available ERβ antibodies that have been poorly characterized for specificity and sensitivity. Third, most of the conclusions have been based on a few breast cancer cases. Clearly, more studies are needed to clarify the role of ERα and ERβ in breast cancer.

Role of SERMs as adjuvant therapy and chemoprevention in breast cancer: Because estrogens promote the proliferation of breast cancer cells, several therapeutic approaches have been implemented to block this effect of estrogens on breast tumors. These strategies, including ovarian ablation, antiestrogens, gonadotropin releasing hormone analogs or aromatase inhibitors, work by either decreasing the production of estrogens or blocking the action of estrogens. All of these strategies non-selectively block the action of both ERα and ERβ. The most common approach used clinically to prevent and treat breast tumors are the selective estrogen receptor modulators (SERMs), tamoxifen and raloxifene.

Tamoxifen is a non-steroidal triphenylethylene derivative that is the prototype SERM, because it exhibits antagonistic action in some tissues, such as the breast, but has agonist actions in other tissues such as the endometrium and bone. Tamoxifen has been extensively studied for its clinical effectiveness as an adjuvant therapy to reduce the recurrences of breast tumors in women with estrogen receptor-positive breast cancer. Five years of tamoxifen therapy reduces the risk of recurrences by 42%, mortality from breast cancer by 22% and a second contralateral primary breast tumor. Approximately, ⅔ of ER positive breast tumors respond to tamoxifen, whereas very little evidence indicates that women with ER negative tumors benefit from adjuvant tamoxifen. Most recently, the U.S. Breast Cancer Prevention Trial (BCPT) demonstrated that tamoxifen reduces the risk of primary invasive breast cancer by 49% in women considered to be at high risk for breast cancer. These studies demonstrate that tamoxifen is a first-line effective adjuvant therapy in women with a history of breast cancer and is an effective chemoprevention agent for women who are high risk for developing breast cancer.

Raloxifene is a member of the benzothiophene class of SERMs that has recently been approved for the prevention and treatment of osteoporosis. Raloxifene has not been evaluated for effectiveness as an adjuvant therapy for women with breast cancer. However, the Multiple Outcomes of Raloxifene (MORE) trial evaluated the effect of raloxifene on preventing breast cancer. The MORE trial was a randomized, placebo-controlled three-year study of 7705 postmenopausal women who have osteoporosis. In the MORE trial, 13 cases of breast cancer were found among the 5129 women in the raloxifene treatment group versus 27 among the 2576 women who received placebo (RR=0.24) after a median follow-up of 40 months. Like tamoxifen, raloxifene is effective at reducing the incidence of estrogen receptor positive tumors, but not estrogen receptor negative tumors. Additional evidence for a role of estrogens in promoting breast cancer comes from a recent study that showed raloxifene only prevents breast cancer in postmenopausal women that have detectable levels of serum estradiol.

Structure of Estrogens Receptors: The fact that SERMs only work on ER positive tumors indicates that they need to interact with estrogen receptors in order to exert its protective effects on the breast. There are two known estrogen receptors, ERα and ERβ, which are members of the steroid nuclear receptor superfamily. ERα was first cloned in 1986, and surprisingly about 10 years later a second ER was discovered, and named ERβ. ERα contains 595 amino acids, whereas ERβ contains 530 amino acids. Both receptors are modular proteins made up of three distinct domains. The amino-terminus domain (A/B domain) is the least conserved region, exhibiting only a 15% homology between ERα and ERβ. This domain harbors an activation function (AF-1) that can activate gene transcription activation in the absence of estradiol. The central region of ERs contains two zinc finger motifs that bind to an inverted palindromic repeat sequence separated by three nucleotides located in the promoter of target genes. The DNA binding domain (DBD) in ERα and ERβ are virtually identical, exhibiting 95% homology. The carboxy-terminus domain contains the ligand binding domain (LBD), which carries out several essential functions. The LBD contains a region that forms a large hydrophobic pocket where estrogenic compounds bind, as well as regions involved in ER dimerization. The LBD also contains a second activation function (AF-2) that interacts with coregulatory proteins. AF-2 is required for both estrogen activation and repression of gene transcription. The LBDs of ERα and ERβ are only about 55% homologous. The striking differences in the amino acid composition of the ERα and ERβ LBDs may have evolved to create ERs that have distinct transcriptional roles. This would permit ERα and ERβ to regulate the activity of different genes and to elicit different physiological effects. This notion is supported by studies of ERα and ERβ knockout mice. For example, the ERα knockout mice have primitive mammary and uterine development, whereas the ERβ knockout mice develop normal mammary glands and uterus. These observations demonstrate that only ERα is required for the development of these tissues. Furthermore, the inventor has found that ERα is more effective than ERβ at activating genes, whereas ERβ is more effective than ERα at repressing gene transcription.

Mechanisms of action of estrogens: Estrogens can activate or repress gene transcription. There are two characterized pathways for activation of gene transcription, the classical ERE (estrogen response element) pathway and the AP-1 pathway. There are at least three essential components necessary for estrogens to regulate the transcription of genes: the ERs (ERα and/or ERβ), the promoter element in target genes and coregulatory proteins. The binding of estradiol to the ER leads to a conformational change, which results in several key steps that initiate transcriptional pathways. First, the interaction of E₂ with ER leads to the dissociation of chaperone proteins; this exposes the ER's dimerization surface and DNA binding domain. Loss of the chaperone proteins allows the ERs to dimerize and bind to an ERE in the promoter region of a target gene.

Second, the binding of E₂ moves helix 12 of the ER's LED to create a surface that assembles the AF-2 function of the ER. The AF-2 consists of a conserved hydrophobic pocket comprised of helices 3, 5 and 12 of the ER, which together form a binding surface for the p160 class of coactivator proteins (coactivators), such as steroid receptor coactivator-1 (SRC-1) or glucocorticoid receptor interacting protein 1 (GRIP 1). Coactivators (also known as “coregulatory”) contain several repeat amino acid motifs comprised of LXXLL, which project into hydrophobic cleft surrounded by the AF-2's helices. The coactivators possess histone acetylase activity. It is thought that gene activation occurs after the ERs and coactivator proteins form a complex on the ERE that causes the acetylation of histone proteins bound to DNA. The acetylation of histones changes the chromatin structure so that the ER/coregulator complex can form a bridge between the ERE and basal transcriptional proteins that are assembled at the TATA box region of the target gene to initiate gene transcription.

Effect of SERMs on the ERE pathway: Unlike estrogens, SERMs do not activate the ERE pathway. Instead, the SERMs competitively block the effects of estrogens on the ERE pathway. Like estrogens, SERMs bind to ERα and ERβ with high affinity and cause the dissociation of chaperone proteins, ER dimerization and binding of ERs to the ERE. Thus, the antagonist action of SERMs occurs at a step distal to the binding of the ER to the promoter region. The molecular mechanism of the antagonist action of the SERMs has been clarified by the crystallization of the ERα and ERβ LBDs. It is clear from the structure of the ER LBDs that E₂, tamoxifen and raloxifene bind to the same binding pocket. However, tamoxifen and raloxifene contain a bulky side-chain that is absent in E₂. The ER x-ray structures have revealed that the bulky side chain of SERMs obstructs the movement of the LBD, which prevents the formation of a functional AF-2 surface. Remarkably, when a SERM binds to ERα, a sequence (LXXML) in helix 12, which is similar to the LXXLL motif, interacts with the hydrophobic cleft of the AF-2 surface to occlude the coactivator recognition site. Thus, unlike estrogens, SERMs do not create a functional AF-2 surface; this prevents the binding of coactivators. Because the coactivator proteins do not bind to the AF-2 surface in the presence of SERMs, the activation pathway is abruptly halted. Instead of recruiting coactivator, ERs liganded with SERMs recruit corepressors, such as N-CoR.

These studies demonstrated that the antagonist properties of SERMs are due to at least three factors. First, SERMs bind to the same binding pocket as estrogens and competitively block their binding to the ERs. Second, SERMs prevent ER from interacting with coactivator proteins that are required for transcriptional activation of the ERE pathway. Third, SERMs recruit corepressors, which prevent transcriptional activation of genes. These actions of SERMs most likely explain how raloxifene and tamoxifen act as antagonists in breast cells to inhibit development of breast cancer.

SERMs are also more effective than E₂ at activating genes with an AP-1 element. In fact, E₂ is an antagonist of SERM-mediated activation of AP-1 elements. It has been postulated that SERMs exhibit agonistic actions in tissues, such as the bone and endometrium by activating the AP-1 pathway. Interestingly, SERMs are more potent at activating the AP-1 pathway in the presence of ERβ, which indicates that SERMs will trigger the AP-1 pathway more efficiently in tissues that are rich in ERβ. The role of the AP-1 pathway in estrogen-mediated breast carcinogenesis is unclear, because estrogens are much weaker at activating the AP-1 pathway compared to SERMs. However, it has been proposed that the AP-1 pathway may be involved in resistance to tamoxifen in breast tumors.

In accordance with aspects of the present invention, studies have been performed, which demonstrate that: ERβ is weaker than ERα at activating ERE-tkLuc; ERβ is more effective than ERα at repressing the TNF-RE-tkLuc; and that ERβ inhibits ERα-mediated transcriptional activation of ERE-tkLuc. Detailed experiments are discussed in the Examples section hereinafter.

### Manufacture of Estrogen Receptor Beta-Modulating Compositions

Total synthesis of nyasol can be effected as shown in Scheme 1 below:

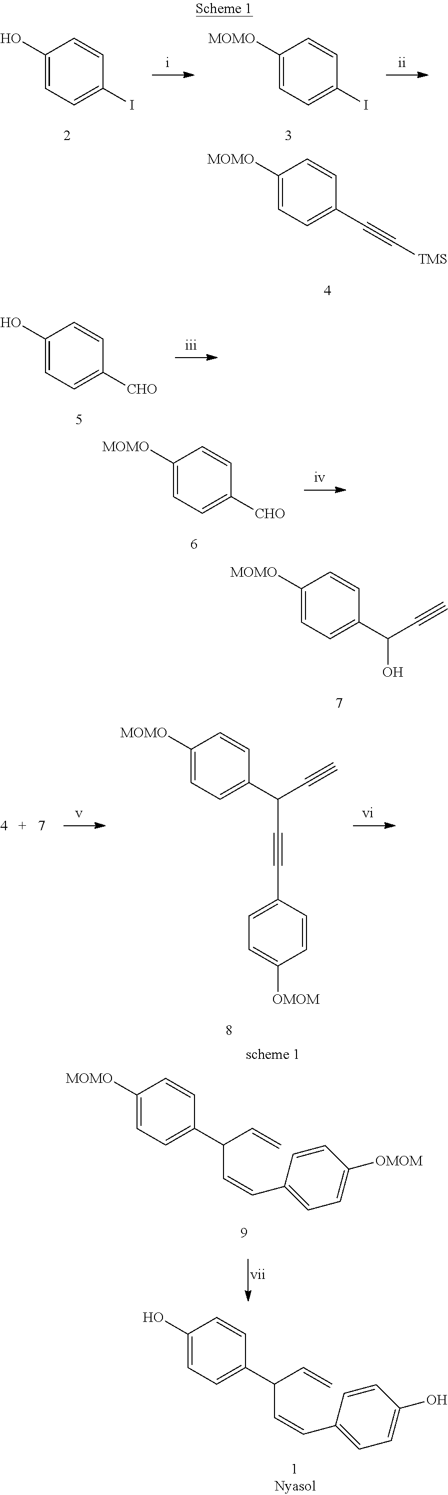

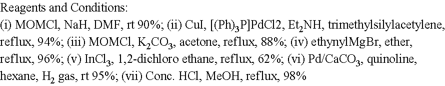

According to the foregoing scheme I, one method of preparing nyasol comprises:

(a) protecting the hydroxy group of 4-iodophenol with MOMCl to form a protected intermediate:

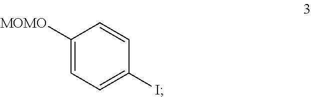

(b) contacting 3 with trimethylsilylacetylene in the presence of bis[triphenylphosphine]palladium dichloride and CuI in diethylamine to form:

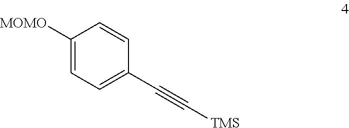

(c) reacting 4-hydroxybenzadehyde with MOMCl to form:

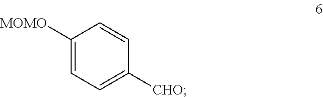

(d) reacting 6 with ethynlymagneciumbromide to form:

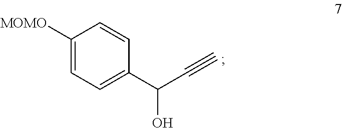

(e) reacting 4 and 7 to form 8:

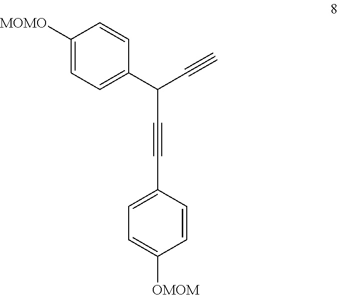

(f) reducing the triple bonds in 8 to form 9:

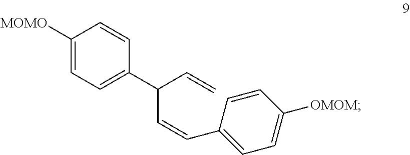

and
(g) removing the MOM protective groups to form 1 (nyasol):

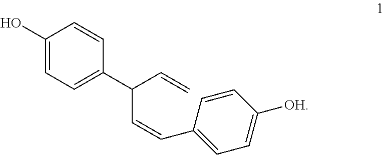

Compounds (b)-(h) can be prepared by selectively blocking one of the hydroxy groups, coupling to the unblocked hydroxy group the appropriate conjugate group and deblocking the protected hydroxy group. In some alternative embodiments, starting materials 2 and 5 may be protected with different hydroxy blocking groups which may be removed under different conditions. Reaction then progresses by selectively removing one or the other protecting groups and coupling the resulting deprotected hydroxy group with an appropriate reagent then removing the remaining protecting group to produce the appropriate compound (b), (c), (d), (e), (f), (g) or (h). Alternatively, the reaction scheme II may be followed:

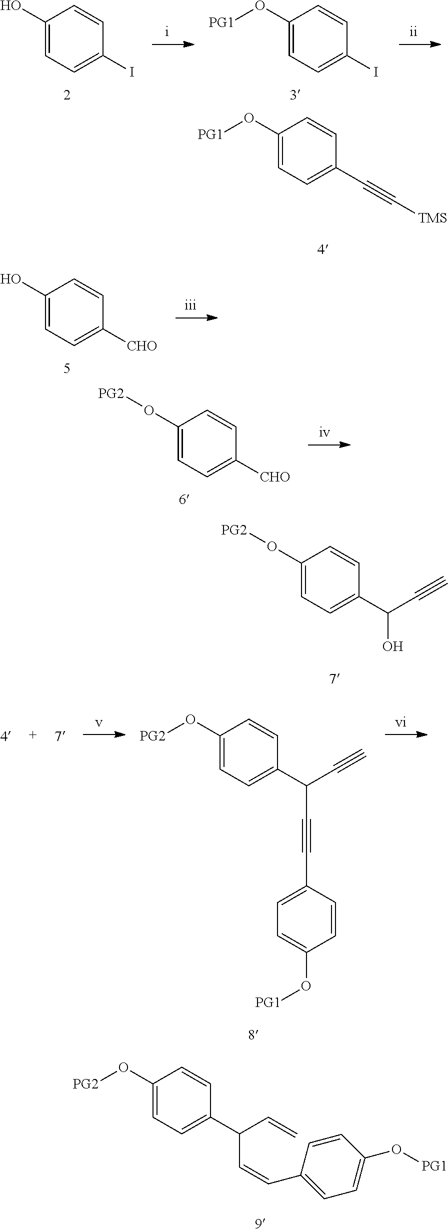

Wherein PG1 is a first protecting group removable under a first set of conditions and PG2 is a second protecting group removable under a second, distinct set of conditions. Reaction may then proceed under one of the following Schemes IIIa or IIIb:

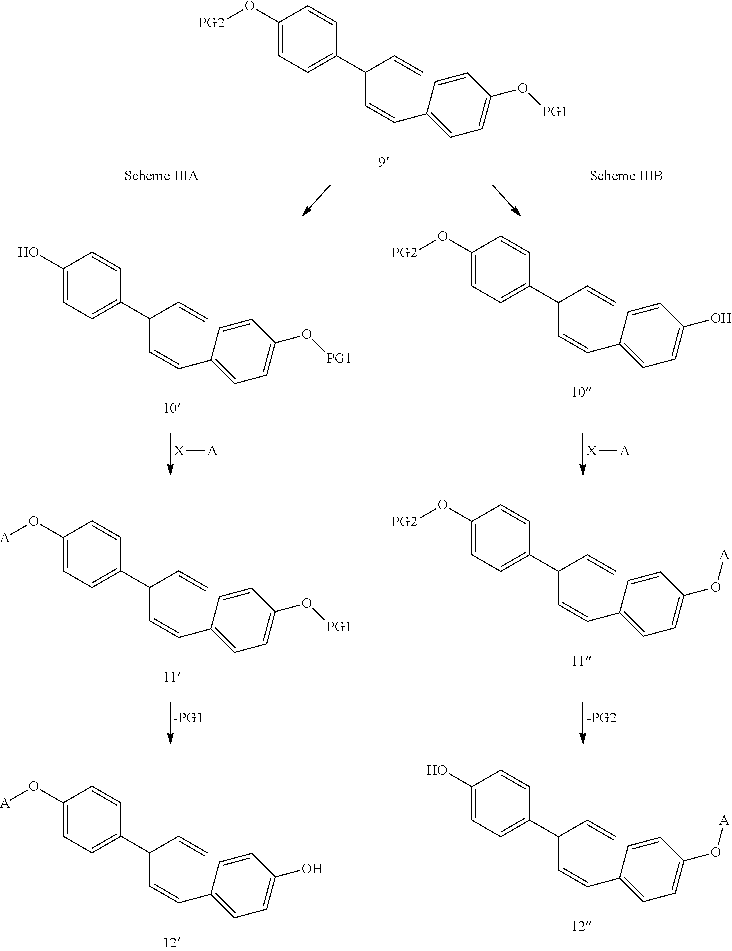

Wherein X is a leaving group and A is the conjugate group corresponding to one of the conjugate groups in compound (b), (c), (d), (e), (f), (g) or (h). Suitable protecting groups are known, as are the differential methods of removing said protecting groups. Suitable conjugating reagents X-A are also known.

### Pharmaceutical Compositions

Pharmaceutical compositions described herein contain one or more compounds described herein:

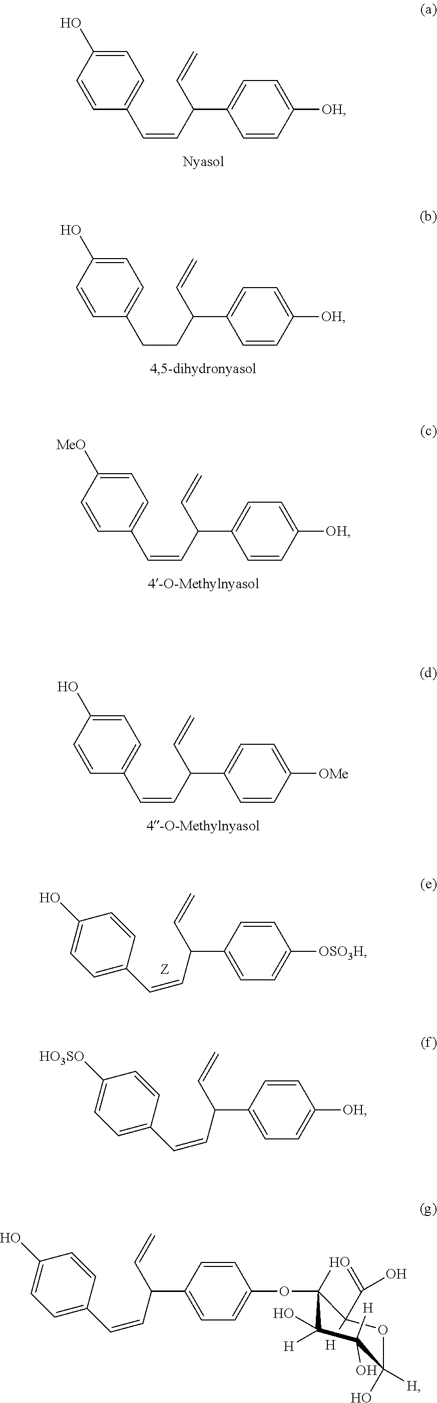

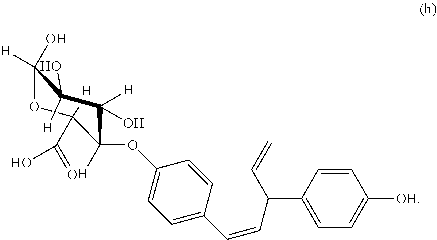

A pharmaceutical composition comprising one or more of (a), (b), (c), (d), (e), (f), (g) and (h) may be prepared as above in either solution or dried form. In a solution form, a pharmaceutical composition comprising one or more of (a), (b), (c), (d), (e), (f), (g) and (h) may be administered in the form a flavored or unflavored tea. In some embodiments some flavoring, e.g. sweetening, may be desirable to counteract the bitter flavor of the pharmaceutical composition comprising one or more of (a), (b), (c), (d), (e), (f), (g) and (h). Solutions can also be prepared in tea or elixir forms. Again, flavoring, such as sweetening may be desirable. Taste-masking may be employed to improve patient acceptance of the pharmaceutical composition.

A pharmaceutical composition comprising one or more of (a), (b), (c), (d), (e), (f), (g) and (h) may be formulated as an orally-available form, such as in a capsule, tablet, caplet, etc. A capsule may be prepared by measuring a suitable amount of the pharmaceutical composition comprising one or more of (a), (b), (c), (d), (e), (f), (g) and (h) into one or more gelatin capsule shells and assembling the capsule(s). Tablets and caplets may be prepared by combining the pharmaceutical composition comprising one or more of (a), (b), (c), (d), (e), (f), (g) and (h) with one or more binders and optionally one or more disintegrants. Tablets, caplets, capsules, etc. may be coated, e.g. with an enteric coating, to prevent stomach upset.

A pharmaceutical composition comprising one or more of (a), (b), (c), (d), (e), (f), (g) and (h) may be combined with one or more gelling agents and inserted into a gel capsule. Alternatively, pharmaceutical composition comprising one or more of (a), (b), (c), (d), (e), (f), (g) and (h) may be combined with a gelling agent and optionally one or more flavoring agents for oral administration as an edible gel or a non-flavored variant may be administered as a rectal suppository gel or gel capsule.

A unit dose of a composition comprising one or more of (a), (b), (c), (d), (e), (f), (g) and (h) is may contain 1 mg to about 10 g of one or more of (a), (b), (c), (d), (e), (f), (g) and (h). In some embodiments, the unit dose will contain about 1 mg to about 10 mg, about 1 mg to about 100 mg, about 1 mg to about 1000 mg (1 g), about 1 mg to about 10000 mg (10 g) of one or more of (a), (b), (c), (d), (e), (f), (g) and (h). In some embodiments, the unit dose contains about 10 mg to about 100 mg, about 10 mg to about 1000 mg or about 10 mg to about 10000 mg of one or more of (a), (b), (c), (d), (e), (f), (g) and (h). In some embodiments, the unit dose contains about 100 mg to about 5000, about 100 mg to about 2500 mg, about 100 mg to about 2000 mg, about 100 mg to about 1500 mg, about 100 to about 1000, about 100 to about 800 mg of composition comprising one or more of (a), (b), (c), (d), (e), (f), (g) and (h), or the equivalent thereof.

Pharmaceutical compositions comprising one or more of (a), (b), (c), (d), (e), (f), (g) and (h) provide ERβ-selective estrogenic activation of genes under control of the estrogen response element (ERE). Accordingly, in some cells contacting said cells comprising an ERE and ERβ with composition comprising one or more of (a), (b), (c), (d), (e), (f), (g) and (h) gives rise to stimulation of a gene under control of the ERE. In an in vitro cell system, ERE-mediated activation by a composition comprising one or more of (a), (b), (c), (d), (e), (f), (g) and (h) leads to expression of a gene that is operatively linked to the ERE. In particular embodiments, estrogenic interaction of an ER with an ERE linked to the minimal thymidine kinase promoter and the luciferase gene gives rise to enhanced luciferase expression. Thus, a composition comprising one or more of (a), (b), (c), (d), (e), (f), (g) and (h) of the present invention may be used to identify ERα+ cell lines, ERβ+ cell lines and/or ERα+/ERβ+ cell lines having an ERE-containing promoter operatively linked to a reporter gene, such as luciferase. Compositions comprising one or more of (a), (b), (c), (d), (e), (f), (g) and (h) may also be used as assay reagents, including standards, for identifying compounds having estrogenic effects in ER+ cell lines.

In one such assay method, an a composition comprising one or more of (a), (b), (c), (d), (e), (f), (g) and (h) is first prepared at a known activity or concentration.

In general the ER+ cells are contacted with the a composition comprising one or more of (a), (b), (c), (d), (e), (f), (g) and (h) and a signal relating to estrogenic activity is recorded. In particular, an ER+ cell has a reporter gene under the control of an ERE. This ER+ cell is contacted with a composition comprising one or more of (a), (b), (c), (d), (e), (f), (g) and (h) of the invention, which gives rise to a reporter signal in proportion to the amount of a composition comprising one or more of (a), (b), (c), (d), (e), (f), (g) and (h) added. This step may be carried out with multiple samples at the same a composition comprising one or more of (a), (b), (c), (d), (e), (f), (g) and (h) concentration, at different a composition comprising one or more of (a), (b), (c), (d), (e), (f), (g) and (h) concentrations, or both. As an example, nine samples may be tested: the first three at a first concentration, the next three at a concentration that is a half log greater than the first, and the next three at a concentration a whole log greater than first. The reporter signals are then observed and recorded, and the resulting data points (a composition comprising one or more of (a), (b), (c), (d), (e), (f), (g) and (h) concentration versus reporter signal strength) are fitted to a standard curve by a conventional curve-fitting method (e.g. least squares).

To evaluate the estrogenic effect of a candidate compound, a candidate compound is contacted with E+ cells having the reporter gene under control of the ERE. The reporter gene signal is observed and compared to the standard curve to quantitate the candidate compound's relative estrogenic effect.

The ER+ cell line used in the foregoing method may be a cell line that naturally expresses ER, e.g. a human-derived ER+ breast cell carcinoma cell line. In some embodiments, the ER+ tissue is an immortalized human cell line, e.g. an immortalized bone marrow or breast cell line. Exemplary cell lines include human monocyte, osteoblast, malignant breast carcinoma and immortalized epithelial breast cell lines. Particular cell lines that may be mentioned include U937, U2OS, MDA-MB-435 and MCF-7 cell lines. Other ER+ cell lines, including immortalized cell lines, may also be used. Alternatively, the ER+ cell line may be a cell line that does not naturally express ER, such as a bacterial cell line, that has been transformed with an ER expression vector.

The ER+ cell line is transformed with a vector having a promoter containing an ERE that controls a reporter gene. For example, the vector may be a viral vector containing ERE, a minimal thymidine kinase promoter (tk) and a luciferase gene (Luc). An exemplary ERE-tk-Luk construct is depicted in SEQ ID NO:1, where the ERE is represented by nucleotides 1-, tk is represented by nucleotides nn-, and Luk is represented by nucleotides mm-. The construct is transfected into the target cell by known methods and expression of the ER-ERE-tk-Luk system is confirmed by e.g. performing the foregoing assay on putative ER+ cells in the presence of known quantities of E₂. Other methods of verifying successful transformation of ER+ cells include immunostaining with known ER antibodies.

The ERE-containing promoter is a DNA containing an ERE sequence and a promoter sequence. The promoter sequence is an art-recognized promoter sequence, such as the minimal thymidine kinase (tk) promoter sequence. (See SEQ ID NO:1, nucleotides nn-). Other ERE-containing promoters are possible and are within the scope of the instant invention. The ERE and promoter sequence operate together to control expression of the reporter gene. As described herein, the estrogenic composition (a composition comprising one or more of (a), (b), (c), (d), (e), (f), (g) and (h), for example) binds to the ER, giving rise to ER dimer and forming the AF-2 surface. The ER dimer then binds to the ERE, activating the gene under control of the promoter. In some embodiments, the ERE is directly upstream of (5′- to) the promoter, to which it is directly ligated. As an example, the ERE-tk promoter construct is shown in SEQ ID NO: 1, nucleotides 1-nn-1.

The reporter gene is a gene which, when expressed, gives rise to a detectable signal. The luciferase gene is a suitable reporter gene because it gives rise to the protein luciferase, which generates a detectable light signal in the presence of a single reagent, luciferin. In particular, the cDNA of the luciferase gene is expressed to produce the 62 kDa enzymatic protein, luciferase. The luciferase enzyme catalyzes the reaction of luciferin and ATP in the presence of Mg²⁺ and oxygen to form oxyluciferin, AMP, pyrophosphate (PPi) and emitted light. The emitted light is yellow-green (560 nm), and may easily be detected using a standard photometer. Because ATP, O₂ and Mg²⁺ are already present in cells, this reporter gene only requires addition of the reagent luciferin to produce a detectable signal, and is especially well-suited for use in assays of the present invention. Other reporter genes that may be mentioned as being available in the art include chloramphenicol transacetylase (CAT), neomycin phosphotransferase (neo) and beta-glucuronidase (GUS).

In some assay methods of the invention, it is useful to further characterize the standard a composition comprising one or more of (a), (b), (c), (d), (e), (f), (g) and (h) by comparison with one or more estrogenic compounds, SERMs, etc. Such assay methods are performed essentially as described above, making the proper substitutions of standard estrogenic compound and/or SERMs for a composition comprising one or more of (a), (b), (c), (d), (e), (f), (g) and (h) in the appropriate parts of the method.

Compositions comprising one or more of (a), (b), (c), (d), (e), (f), (g) and (h) according to the present invention also repress gene expression by the TNF RE-mediated pathway. In some cases, compositions comprising one or more of (a), (b), (c), (d), (e), (f), (g) and (h) repress gene expression in vitro, especially in cells having a reporter gene (e.g. the luciferase gene, Luc) under control of a TNF RE. In some cases, compositions comprising one or more of (a), (b), (c), (d), (e), (f), (g) and (h) repress expression of TNF-α, which is a cytokine produced primarily by monocytes and macrophages. This cytokine is found in synovial cells and macrophages in various tissues, and has been strongly implicated in rheumatoid arthritis (RA). TNF-α is also expressed in other inflammatory diseases, and also as a response to endotoxins from bacteria. As repressors of TNF expression via the TNF RE repressor pathway, compositions comprising one or more of (a), (b), (c), (d), (e), (f), (g) and (h) are of interest in the treatment of inflammatory disorders associated with elevated levels of TNF.

In some embodiments of the invention, a cell line is prepared, which expresses one or both of ERα and ERβ as well as a reporter gene under control of TNF RE. The TNF RE is generally upstream of (5′- to) the reporter gene, and signal detection is carried out as previously described herein. The sequence of DNA having a reporter gene, in this case luciferase gene, under control of TNF RE is set forth in SEQ ED NO:2. Nucleotides 1-correspond to the TNF RE, while nucleotides nn- corresponds to the luciferase gene.

The foregoing cell TNF RE-containing cell system further contains one or more copies of an ER gene—i.e. ERα, ERβ or both. The ER+ cell line used in the foregoing method may be a cell line that naturally expresses ER, e.g. a human-derived ER+ breast cell carcinoma cell line. In some embodiments, the ER+ tissue is an immortalized human cell line, e.g. an immortalized bone marrow or breast cell line. Exemplary cell lines include human monocyte, osteoblast, malignant breast carcinoma and immortalized epithelial breast cell lines. Particular cell lines that may be mentioned include U937, U2OS, MDA-MB-435 and MCF-7 cell lines. Other ER+ cell lines, including immortalized cell lines, may also be used. Alternatively, the ER+ cell line may be a cell line that does not naturally express ER, such as a bacterial cell line, that has been transformed with an ER expression vector.

In the presence of a predetermined amount of luciferin, and in the absence of an estrogenic compound, e.g. E₂ or a compositions comprising one or more of (a), (b), (c), (d), (e), (f), (g) and (h), the cell system emits a yellow light (560 nm) at an intensity, called the “control intensity” or the “baseline intensity”. Light emission at 560 nm is conveniently quantified in optical density units (O.D.₅₆₀ₙₘ). Upon addition of an estrogenic compound, e.g. E₂ or one of the a composition comprising one or more of (a), (b), (c), (d), (e), (f), (g) and (h)s, the intensity of 560 nm light emissions is attenuated as compared to the control. Remarkably, in the presence of a SERM, such as tamoxifen or raloxifene, luciferase expression increases and 560 nm light emission intensity also increases. Thus, compositions comprising one or more of (a), (b), (c), (d), (e), (f), (g) and (h) are capable of inducing an estrogenic TNF RE-controlled repression of gene expression.

The TNF RE-containing cell system can be used in an assay method according to the invention. In the inventive assay methods, the attenuation of luciferase activity (i.e. decreased emission of 560 nm light), correlates with increased estrogenic activity, whereas activation of luciferase activity (i.e. increased emission at 560 nm), correlates with anti-estrogenic activity. Standard curves may be prepared using known quantities of the a composition comprising one or more of (a), (b), (c), (d), (e), (f), (g) and (h)s, as described herein. Such standard curves may be further augmented by using other known estrogenic or anti-estrogenic standards, such as E₂ or some other known estrogenic compound, and/or an anti-estrogenic SERM such as tamoxifen or raloxifene.

Cells from the transformed E+ cell line are then exposed to a candidate compound, the luciferase signal observed, and the signal compared to the previously prepared standard curve(s), as described herein. A compound that causes an increase of luciferase activity as compared to control (baseline), will be characterized as an anti-estrogenic SERM, whereas a compound that causes a decrease in luciferase activity versus control will be classified as estrogenic. The estrogenic or anti-estrogenic effect can then be quantified by comparing the degree of luciferase expression decrease or increase against the decrease brought about by the a composition comprising one or more of (a), (b), (c), (d), (e), (f), (g) and (h), and optionally the respective signal decrease or increase brought about by E₂, tamoxifen and/or raloxifene.

Pharmaceutical compositions comprising one or more of (a), (b), (c), (d), (e), (f), (g) and (h) of the present invention also antagonize the interaction of E₂-ER with ERE. In particular, it has been shown in that extracts of Astragalus membranaceus antagonize the activation of ERE-tk-Luc by E₂ by directly interacting with ERβ and ERα. As antagonists of E₂-ER activation of ERE-controlled genes, the a composition comprising one or more of (a), (b), (c), (d), (e), (f), (g) and (h) compositions are considered to be similar in effect to tamoxifen, possessing prophylactic, palliative and/or anti-proliferative activity against breast cancer and uterine cancer.

Embodiments disclosed herein provide in vivo estrogenic methods of using the inventive compositions. In general, in vivo methods comprise administering to a subject an amount of the composition comprising one or more of (a), (b), (c), (d), (e), (f), (g) and (h) sufficient to bring about an estrogenic effect in the subject. The in vivo methods will give rise to estrogenic ERE-controlled gene activation, TNF RE-controlled gene repression (e.g. TNF-α repression), or both. Thus, the in vivo methods will give rise to varied positive phenotypic effects in vivo.

The subject may be a mammal, such as a mouse, rat, rabbit, monkey, chimpanzee, dog, cat or a sheep, and is generally female. The subject may also be human, especially a human female. In some embodiments, the subject is a post-menopausal or post-oophorectomic female, and is in need of estrogenic therapy. In such case, the subject may be suffering from climacteric symptoms, such as hot flashes, insomnia, vaginal dryness, decreased libido, urinary incontinence and depression. In other such cases, the subject may be susceptible to, or suffering from, osteoporosis. Suitable in vivo methods include treatment and/or prevention of medical indications that are responsive to estrogen replacement therapy.

Administration of the compositions according to the present invention will be via a commonly used administrative route so long as one or more of the compositions comprising one or more of (a), (b), (c), (d), (e), (f), (g) and (h) is available to target tissue via that route. Some administrative routes that may be mentioned include: oral, nasal, buccal, rectal, vaginal and/or topical (dermal). Alternatively, administration may be by orthotopic, intradermal, subcutaneous, intramuscular, intraperitoneal or intravenous injection. Such compositions would normally be administered as pharmaceutically acceptable compositions, described supra.

Treatment (and its grammatical variants—e.g. treat, to treat, treating, treated, etc.) of a disease, disorder, syndrome, condition or symptom includes those steps that a clinician would take to identify a subject to receive such treatment and to administer a composition of the invention to the subject. Treatment thus includes diagnosis of a disease, syndrome, condition or symptom that is likely to be ameliorated, palliated, improved, eliminated, cured by administering the estrogenic compositions comprising one or more of (a), (b), (c), (d), (e), (f), (g) and (h) to the subject. Treatment also includes the concomitant amelioration, palliation, improvement, elimination, or cure of the disease, disorder, syndrome, condition or symptom. In some embodiments, treatment implies prevention or delay of onset of a disease, disorder, syndrome, condition or symptom (i.e. prophylaxis), prevention or delay of progression of a disease, disorder, syndrome, condition or symptom, and/or reduction in severity of a disease, disorder, syndrome, condition or symptom. In the case of neoplastic growth in particular, treatment includes palliation, as well as the reversal, halting or delaying of neoplastic growth. In this regard, treatment also includes remission, including complete and partial remission. In the case of climacteric symptoms, treatment includes prevention and palliation of various symptoms.

Prevention (and its grammatical variants) of a disease, disorder, syndrome, condition or symptom includes identifying a subject at risk to develop the disease, disorder, syndrome, condition or symptom, and administering to that subject an amount of the a composition comprising one or more of (a), (b), (c), (d), (e), (f), (g) and (h) sufficient to be likely to obviate or delay the onset of said disease, disorder, syndrome, condition or symptom. In some cases, prevention includes identifying a post-menopausal woman who the clinician believes, applying a competent standard of medical care, to be in need of hormone replacement therapy, and administering a pharmaceutical composition comprising one or more of (a), (b), (c), (d), (e), (f), (g) and (h) of the present invention to the woman, whereby one or more climacteric symptoms is blocked or delayed. In some embodiments, prevention of osteoporosis includes identifying a post-menopausal woman who the clinician believes, applying a competent standard of medical care, to be at risk for developing osteoporosis, and administering a pharmaceutical composition comprising one or more of (a), (b), (c), (d), (e), (f), (g) and (h) of the present invention to the woman, whereby the onset of bone loss is blocked or delayed.

Palliation includes reduction in the severity, number and/or frequency of occurrences of an a disease, disorder, syndrome, condition or symptom. Palliation of climacteric symptoms includes reducing the frequency and/or severity of hot flashes, insomnia, incontinence, depression, etc.

Treatment of osteoporosis includes identifying a person, such as a post-menopausal woman, at risk for bone loss, and administering a pharmaceutical composition comprising one or more of (a), (b), (c), (d), (e), (f), (g) and (h) of the present invention to the woman, whereby bone loss is reduced in severity, delayed in onset, or prevented. In some embodiments, treatment of osteoporosis can also include addition of bone mass.

Additional embodiments disclosed herein provide methods of making the pharmaceutical composition comprising one or more of (a), (b), (c), (d), (e), (f), (g) and (h). The invention specifically provides a method of making an inventive estrogenic pharmaceutical composition comprising one or more of (a), (b), (c), (d), (e), (f), (g) and (h). The method includes obtaining a quantity of plant matter from a plant of the species Astragalus membranaceus. optionally comminuting the plant matter, contacting said plant matter with an extraction medium, and separating the plant matter from the extraction medium.

Extracts of Astragalus membranaceus possess estrogenic activity, meaning that they are characterized in being able to bring about estrogenic effects in subjects, particularly peri- and post-menopausal women. In some embodiments, estrogenic effect means at least one effect selected from the group consisting of: treating or preventing at least one climacteric symptom; treating or preventing osteoporosis; treating or preventing uterine cancer; and treating or preventing cardiovascular disease. In some embodiments, the estrogenic effect includes treating or preventing at least one climacteric symptom selected from the group consisting of: hot flashes, insomnia, vaginal dryness, decreased libido, urinary incontinence, headache and depression. In some embodiments, the estrogenic effect includes treating or preventing osteoporosis. In some embodiments, the estrogenic effect includes treating or preventing hot flashes. In some embodiments, the estrogenic effect includes treating or preventing uterine cancer or breast cancer. In some embodiments, the estrogenic effect does not include increasing the risk of hyperplasia or cancer. In some embodiments, the estrogenic effect does not include increasing the risk of mammary hyperplasia, mammary tumor, uterine hyperplasia, uterine tumor, cervical hyperplasia, cervical tumor, ovarian hyperplasia, ovarian tumor, fallopian tube hyperplasia, fallopian tube tumor. In some embodiments, the estrogenic effect includes reducing the risk of hyperplasia or cancer. In some embodiments, the estrogenic effect includes reducing the risk of mammary hyperplasia, mammary tumor, uterine hyperplasia, uterine tumor, cervical hyperplasia, cervical tumor, ovarian hyperplasia, ovarian tumor, fallopian tube hyperplasia, fallopian tube tumor.

In some embodiments, the plant species are of the plant species Astragalus membranaceus are various cultivars of Astragalus membranaceus

Plant matter means any part or parts of at least one plant from the species Astragalus membranaceus. Plant matter includes the whole plant or any part or parts of the plant, such as the root, bark, wood, leaves, flowers (or flower such as: sepals, petals, stamens, pistils, etc.), fruit, seeds and/or parts or mixtures of any of the foregoing. Plant matter may be fresh cut, dried (including freeze dried), frozen, etc. Plant matter may also be whole or separated into smaller parts. For example, leaves may be chopped, shredded or ground; roots may be chopped or ground; fruit may be chopped, sliced or blended; seeds may be chopped or ground; stems may be shredded, chopped or ground. In particular embodiments of the invention, the plant parts used are the leaves of Astragalus membranaceus.

Pharmaceutical compositions comprising one or more of (a), (b), (c), (d), (e), (f), (g) and (h) of the invention contain at least one extract of an Astragalus membranaceus. An “extract” is a solution, concentrate or residue that results when a plant part is contacted with an extraction solvent under conditions suitable for one or more compounds from the plant to partition from the plant matter into the extraction solvent; the solution is then optionally reduced to form a concentrate or a residue.

Suitable extraction media for the present invention include water and ethyl alcohol. Specifically, where water is the extraction solvent, purified water is suitable. Purified water includes distilled water, deionized water, water for injection, ultrafiltered water, and other forms purified of water. Ethyl alcohol that is employed in some embodiments of the invention is grain ethanol, and in particular undenatured ethanol (e.g. pure grain ethanol, optionally containing some water, e.g. up to about 10% water). In some embodiments, the extraction solvent is water, ethanol, or a mixture thereof. A concentrate or residue may be prepared by reducing (e.g. evaporating or lyophilizing) the extraction solution. Whether in the original extraction solvent, reduced concentrate, or residue form, each of these preparations is considered an “extract” for the purposes of the invention.

A method of producing the composition comprising one or more of (a), (b), (c), (d), (e), (f), (g) and (h) optionally comprises first comminuting the plant matter in order to increase its surface area to volume ratio and to concomitantly increase efficiency of the extraction process. Methods of comminuting plant matter include grinding, chopping, blending, shredding, pulverizing, triturating, etc.

The extraction medium (solvent) is then contacted with the plant matter under conditions suitable for causing one or more phytochemicals, in particular estrogenic phytochemicals, to partition from the plant matter into the extraction medium. Such conditions include, in some cases, heating the extraction medium to a temperature above room temperature, agitation, contact time, etc. Exemplary temperatures for extraction are from about 50° C. to the boiling point of the extraction solvent. Where water is the extraction solvent, the extraction temperature is generally from room temperature to about 100° C.; temperatures of from about 50° C. to about 80° C. are especially suitable, and temperatures of about 75° C. are particularly suitable. In the case of ethanol as an extraction solvent, the extraction temperature is generally from about room temperature to about 78.5° C.; temperatures of from about 50° C. to about 78° C. are especially suitable and a temperature of about 75° C. is particularly suitable. The person of skill in the art will recognize that the proper balance should be drawn between extraction efficiency on the one hand and phytochemical compound stability on the other.

Once the extraction medium and the plant matter are combined, they are optionally agitated to ensure efficient exchange of estrogenic compound from the plant matter into the extraction medium, and are left in contact for a time sufficient to extract a useful amount of phytochemical compound from the plant matter into the extraction medium. After such time has elapsed (e.g. from about 5 min. to about 10 hr., more particularly from about 10 min. to about 5 hr., especially about 30 min. to about 2 hr.), the extraction medium containing the phytochemical compounds is separated from the plant matter. Such separation is accomplished by an art-recognized method, e.g. by filtration, decanting, etc.

A composition according to the invention includes an a composition comprising one or more of (a), (b), (c), (d), (e), (f), (g) and (h) or a composition comprising an a composition comprising one or more of (a), (b), (c), (d), (e), (f), (g) and (h) of the invention. In such embodiments, the inventive composition will optionally contain one or more additional ingredients. Such additional ingredients may be inert or active. Inert ingredients include solvents, excipients and other carriers. Active ingredients include active pharmaceutical ingredients (APIs), including those that exhibit synergistic activity in combination with the a composition comprising one or more of (a), (b), (c), (d), (e), (f), (g) and (h).

### EXAMPLES

The invention may be more fully appreciated with reference to the following illustrative and non-limiting examples.

### Example 1

### Total Synthesis of Nyasol

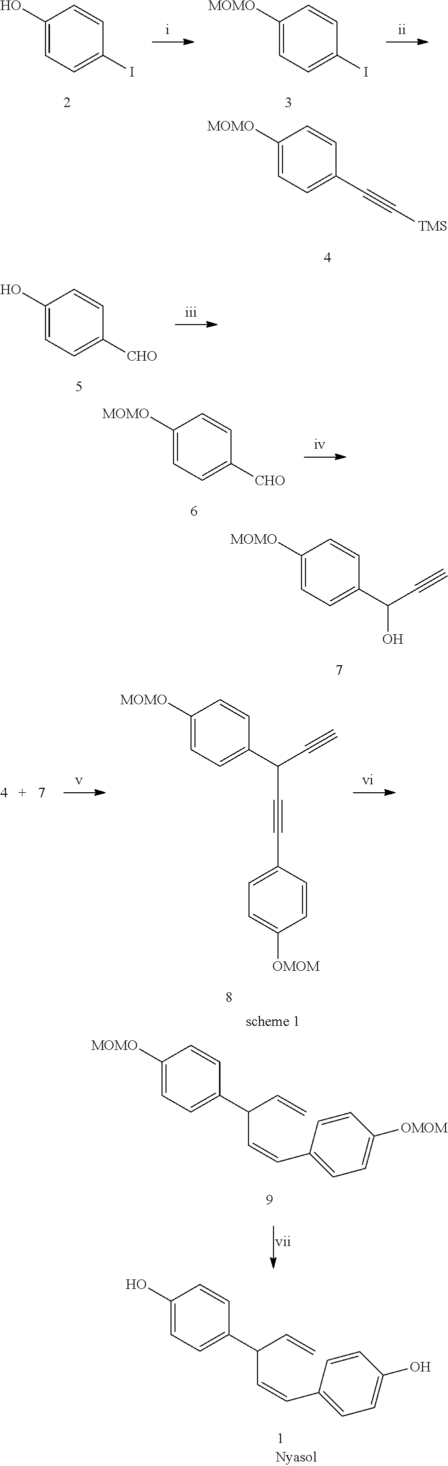

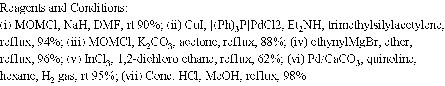

Preparation of 1-iodo-4-(methoxymethoxy)benzene (3):

To a stirred solution of 4-iodophenol (2) (15.0 g, 68.18 mmol) in anhydrous DMF (40.0 mL) was added NaH (2.6 g, 75%). After 30 min added drop wise 7.6 mL MOMCl. Stirring was continued for 3 hr. The reaction was quenched by addition of EtOAc and water. The product was extracted with EtOAc and the combined organic layers were washed with water and dried over anhydrous MgSO₄. Evaporation of the solvent gave 3 as pale yellow liquid (16.2 g, 90%): ¹H-NMR (400 MHz, CDCl₃) δ 7.57 (d, J=9.2 Hz, 2H), 6.82 (d, J=8.8 Hz, 2H), 5.14 (s, 2H), 3.46 (s, 3H); ¹³C NMR (100.00 MHz, CDCl₃) δ 157.33, 138.52, 118.84, 94.60, 84.53, 56.26; m/z (M⁺+H), 264.96.

Preparation of 4-(methoxymethoxy)ethynyl trimethylsilane (4):

To a mixture of 3 (15.28 g, 58.3 mmol), bis[triphenylphosphine]palladium dichloride (920 mg, 1.3 mmol) and CuI (140 mg, 1.4 mmol) in diethylamine (300 ml) was added trimethylsilylacetylene (9.5 mL, 72.2 mmol). The reaction mixture was stirred at room temperature for 6 h under nitrogen and then the solvent was removed under reduced pressure. The residue was extracted into benzene (300 ml) and combined organic layers were dried over MgSO₄ and purified by flash column chromatography (EtOAc/Hexane; 0.5:9.5) to afford 4 as colorless liquid (12.91, 94%): ¹H-NMR (400 MHz, CDCl₃) δ 7.40 (d, J=9.2 Hz, 2H), 6.95 (d, J=8.8 Hz, 2H), 5.16 (s, 2H), 3.46 (s, 3H), 0.23 (s, 9H); ¹³C NMR (100.00 MHz, CDCl₃) δ 157.58, 133.63, 116.71, 116.20, 105.21, 94.48, 92.94, 56.29; m/z (M⁺+H), 235.11.

Preparation of 4-(methoxymethoxy)benzaldhyde (5):

To a stirred solution of 4-hydroxy benzaldehyde (5) (15.03 g, 123.2 mmol) in anhydrous acetone (700.0 mL) was added K₂CO₃ (51.9 g, 376 mmol). After 30 min added drop wise 20.0 mL MOMCl. The reaction mixture was refluxed for 3 h. The reaction was cool to room temperature and filter off the solid. Evaporation of the solvent gave crude product. Flash column chromatography of the reaction mixture (EtOAc/Hexane; 1:9) afforded pale yellow liquid, 4-(methoxymethoxy)benzaldehyde (6) (18.1 g, 88%); ¹H-NMR (400 MHz, CDCl₃) δ 9.90 (s, 1H), 7.84 (d, J=8.8 Hz, 2H), 7.15 (d, J=8.4 Hz, 2H), 5.25 (s, 2H), 3.49 (s, 3H); ¹³C NMR (100.00 MHz, CDCl₃) δ 191.11, 162.42, 132.09, 130.94, 116.49, 94.31, 56.55; m/z (M⁺+H), 166.66.

Preparation of 1-(4-methoxymethoxy-)penylpro-2-yn-1-ol) (7):

To a stirred solution of 6 (15.0 g, 90.2 mmol) in anhydrous ether (300 mL) was added THF solution (0.5 M, 330 mL) of ethynlymagneciumbromide under nitrogen. The reaction mixture was refluxed at 60° C. for 4 h. Then the reaction mixture was cooled to room temperature, neutralizes with 10% HCl, extracted with ether, washed with brine then water and dried over MgSO₄. The solvent is removed under reduced pressure and purified by flash column chromatography (EtOAc/Hexane; 15:85) to afford brown color liquid (16.72 g, 96%); ¹H-NMR (400 MHz, CDCl₃) δ 7.48-7.46 (dd, J=8.8, 2.4 Hz, 2H), 7.05-7.03 (dd, J=8.8, 2.0 Hz, 2H), 5.41 (m, 1H), 5.17 (s, 2H), 3.46 (s, 3H), 2.66 (d, J=2.0 Hz, 1H); ¹³C NMR (100.00 MHz, CDCl₃) δ 157.60, 133.85, 128.26, 116.56, 94.57, 83.87, 74.87, 64.19, 56.20; m/z (M⁺+H), 192.08.

Coupling Reaction of 4 and 7:

To a mixture of 4 (5.57, 29.0 mmol) and 7 (10.99 g, 47.0 mmol) in 1,2-dichloro ethane (40 mL) was added InCl₃ (350.9 mg, 1.5 mmol). The reaction mixture was heated at 60° C. for 4 h. After cooling to room temperature the reaction mixture was partially concentrated then directly subjected to flash column chromatography (EtOAc/Hexane; 8:2) to offered 7 (5.98 g, 61.6%) as pale yellow viscous mass; ¹H-NMR (400 MHz, CDCl₃) δ 7.52-7.49 (dd, J=9.6, 0.8 Hz, 2H), 7.39-7.37 (dd, J=8.8, 2.0 Hz, 2H), 7.05-7.03 (dd, J=8.8, 2.0 Hz, 2H), 6.96-6.94 (dd, J=8.8, 2.4 Hz, 2H), 5.17 (s, 2H), 5.16 (s, 2H), 4.91 (d, J=2.4 Hz, 1H), 3.47 (s, 3H), 3.46 (s, 3H), 2.39 (d, J=2.8 Hz, 1H); ¹³C NMR (100.00 MHz, CDCl₃) δ 157.46, 156.92, 133.39, 131.07, 128.53, 116.69, 116.35, 116.27, 94.70, 94.51, 85.20, 82.77, 81.88, 70.89, 56.29, 56.20, 28.81; m/z (M⁺+H), 337.14.

Controlled Reduction of 8:

To a stirred solution of 7 (2.6 g, 7.7 mmol) in EtOAc (20.0 mL) and anhydrous hexane (200 mL) were added quinoline (96.0 mg, 0.74 mmol) and Pd/CaCO₃ (191.0 mg). The reaction mixture was hydrogenated with H₂ balloons (2 way adaptor) 4 h until the consumption of the starting material was observed through TLC. Filter off the solid and the solvent is removed under reduced pressure and purified by flash column chromatography (EtOAc/Hexane; 30:70) to afford colorless viscous mass (mixture of 4 isomers) (2.51 g, 95%) major compound is 9 (1.7 g, 65%); ¹H-NMR (400 MHz, CDCl₃) δ 7.23 (d, J=8.4 Hz, 2H), 7.15-7.13 (d, J=7.8 Hz, 2H), 7.01-6.96 (m, 1H), 6.55 (d, J=11.2, Hz, 1H), 6.05-5.97 (m, 1H), 5.74-5.70 (dd, 10.0, 11.6, 1H), 5.19 (s, 2H), 5.15 (s, 2H), 4.54-4.09 (m, 1H), 3.65 (s, 3H), 3.47 (s, 3H), 2.04 (s, 1H); ¹³C NMR (100.00 MHz, CDCl₃) δ 156.41, 156.18, 140.86, 136.95, 132.16, 131.10, 130.10, 129.57, 128.85, 128.27, 116.62, 116.36, 116.12, 94.77, 94.65, 56.24, 47.12, 28.81; m/z (M⁺+H), 341.17.

Deprotection of MOM Group:

To a stirred solution of 9 (mixture of isomers) (3.0 g, 8.8 mmol) in anhydrous MeOH (30 mL) was added conc. HCl (4 drops) and the mixture was heated at 65° C. for 1.5 h until the conversion of the starting material into Nyasol was observed through TLC. The solvent was partially evaporated under reduced pressure then extracted with EtOAc and purified by HPLC to afford Nyasol 1 as colorless viscous mass (1.37 g, 65%) and other isomers (0.7 g, 35%); ¹H-NMR (400 MHz, CDCl₃) δ 7.09 (d, J=8.0 Hz, 2H), 7.02-7.13 (dd, J=8.8, 0.8 Hz, 2H), 6.72-6.69 (m, 1H), 6.44 (d, J=11.2, Hz, 1H), 5.98-5.90 (m, 1H), 5.60-5.55 (dd, 10.0, 10.4, 1H), 5.10-5.05 (m, 2H), 4.44-4.40 (m, 1H),; ¹³C NMR (100.00 MHz, CDCl₃) δ 155.36, 154.80, 141.09, 135.31, 131.74, 130.18, 129.54, 129.00, 128.85, 115.53, 115.26, 115.09, 47.05; m/z (M⁺+H), 345.17.

### Example 2

### ERβ is Weaker than ERα at Activating ERE-tkLuc

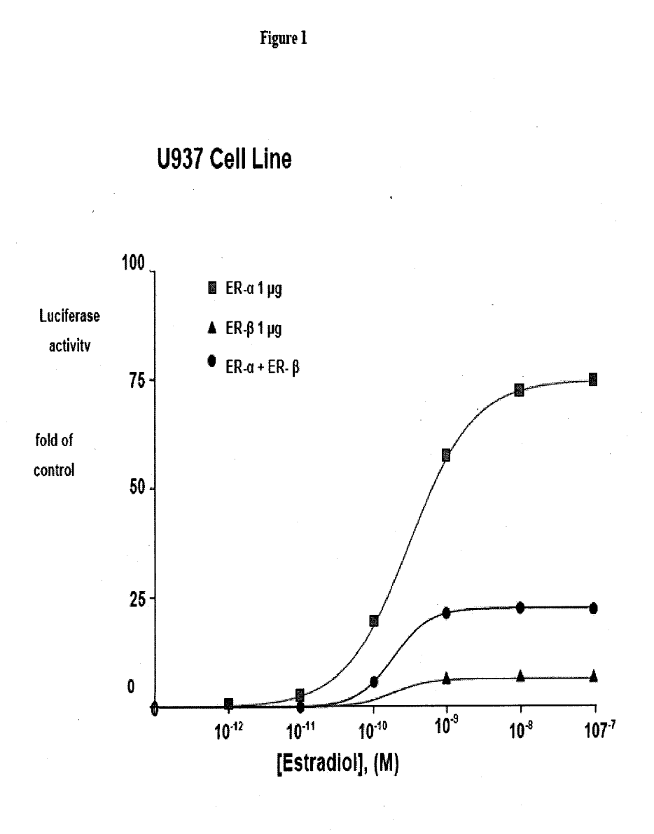

The effects of E2 on transcriptional activation were examined by transfecting a plasmid containing a classical ERE upstream of the minimal thymidine kinase (tk) promoter linked to the luciferase reporter cDNA and an expression vector for ERα or ERβ. E2 produced a 10-fold greater activation of the ERE in the presence of ERα compared to ERβ in human monocytic U937 cells, but the EC50 values were similar. See FIG. 1.

### Example 3

### ERβ is More Effective than ERα at Repressing the TNF-RE-tkLuc

The effects of effects of E2 on ERα and ERβ-mediated transcriptional repression were then compared using the −125 to −82 region of the TNF-α promoter, known as the tumor necrosis factor-response element (TNF-RE). TNF-α produced a 5-10-fold activation of 3 copies of the TNF-RE (−125 to −82) upstream of the tk promoter (TNF-RE tkLuc). E2 repressed TNF-α activation of TNF-RE tkLuc by 60-80% in the presence of ERα and ERβ. However, ERβ was approximately 20 times more effective than ERα at repression (IC50 of 241 pM for ERα versus 15 pM for and ERβ, respectively). It was also found that ERβ is more effective than ERα at repressing the native −1044 to +93 TNF-α promoter. Thus, ERα is much more effective than ERβ at transcriptional activation, whereas ERβ is more effective than ERα at transcriptional repression. In contrast to E2, the antiestrogens, tamoxifen, raloxifene and ICI 182780 produced a 2-fold activation of TNF-RE tkLuc. Furthermore, these antiestrogens abolished the repression induced by E2.

### Example 4

### ERβ Inhibits ERβ-Mediated Transcriptional Activation of ERE-tkLuc

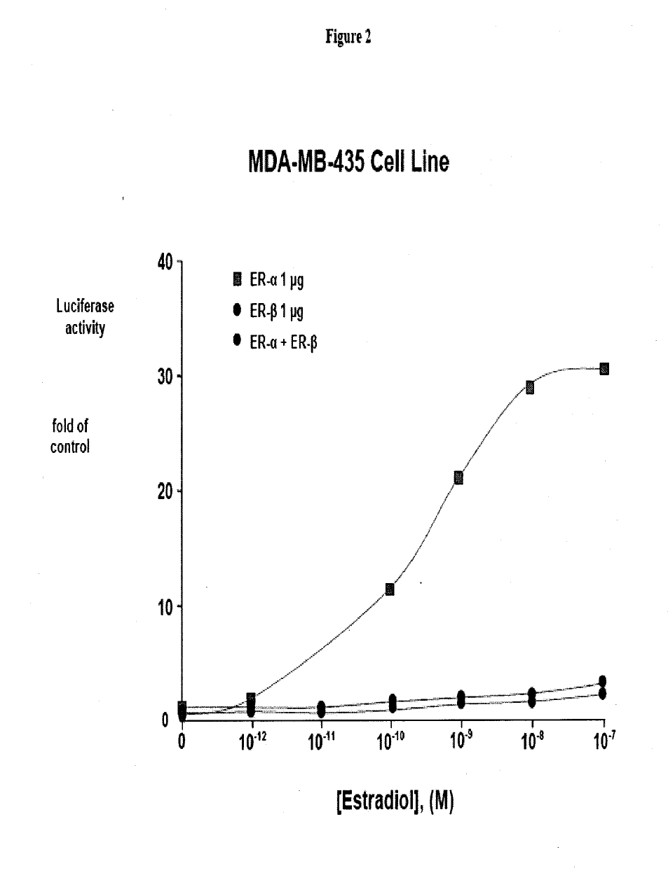

Surprisingly, when ERα or ERβ were coexpressed in U937 cells, the activation by ERα is markedly inhibited. FIG. 1. These data show that ERβ exerts a repressive effect on ERα activation of ERE-tkLuc. Similar results were observed in the breast cancer cell line, MDA-MB-435. See FIG. 2. Other investigators have found a similar repressive effect of ERβ on ERα transactivation in different cell types. These studies indicate that the different activation of ERα and ERβ on ERE-tkLuc and the repressive effect of ERβ on ERα-mediated-transcription are not cell-type specific and results from intrinsic properties of the ERs. The repression of ERα by ERβ requires the formation of an ERα/ERβ heterodimer, because mutations in helix 11 of ERβ that prevent dimerization inhibit its repression activity (data not shown).

### Example 5

### ERβ-Mediated Activation/Repression of Gene Expression with Nyasol (1)

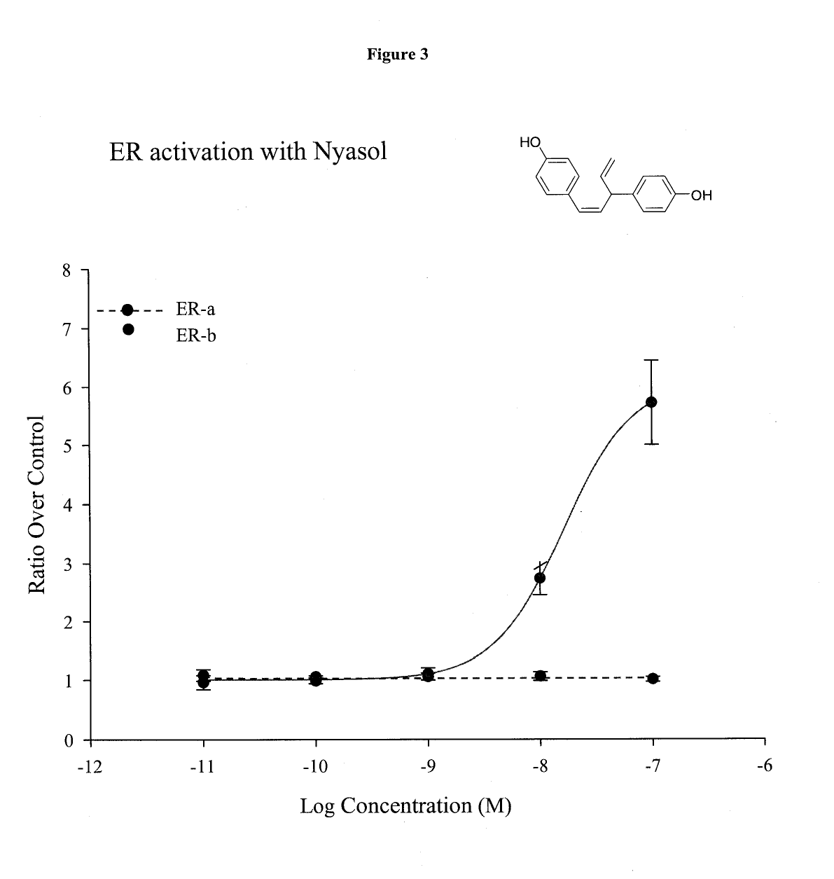

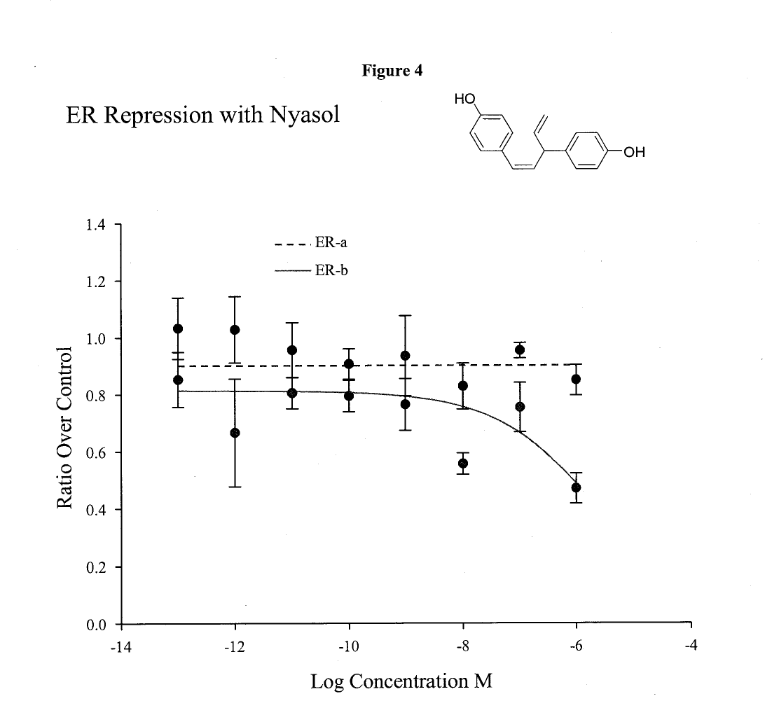

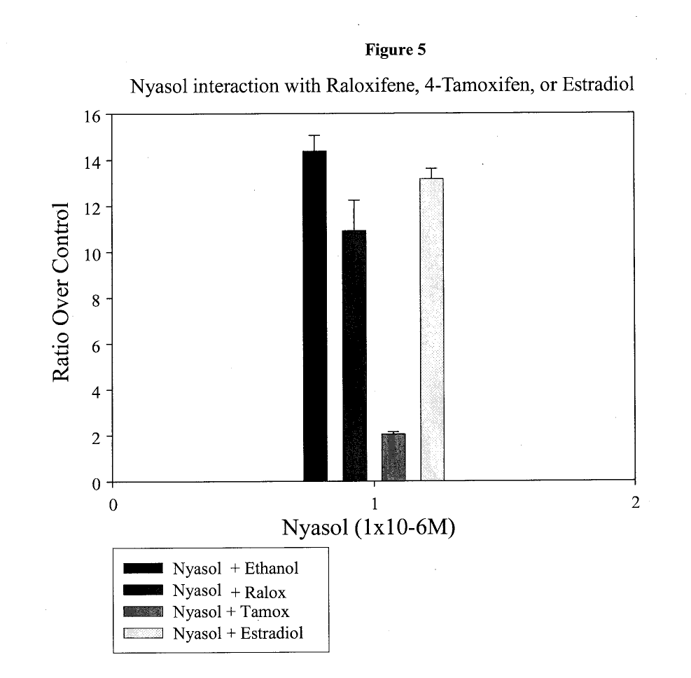

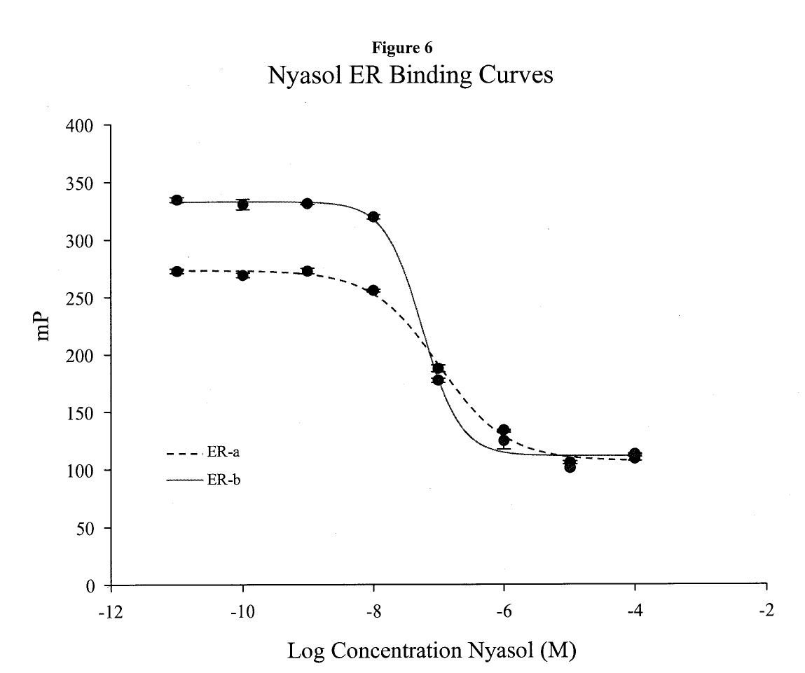

FIG. 3 shows that nyasol selectively activates the ERE in cells transformed with ERE-tkLuc and ERβ through estrogen receptor beta (ERβ). FIG. 4 shows that nyasol selectively represses TNFα-RE linked expression of Luc in vitro. FIG. 5 shows the effect of nyasol on expression of luciferase in ERE-tkLuc-transformed cells coexpressing ERβ. The control was EtOH. Nyasol activated the ERE through the co-expressed ERβ, thereby expressing luciferase. Addition of known ERβ antagonists raloxifene and tamoxifen reduced this activation, while addition of estradiol resulted in activation of the ERE and expression of luciferase. FIG. 6 shows the binding of ERβ and ERα with nyasol. As can be seen, both ERβ and ERα bind nyasol; however the gene expression data in FIGS. 3-5 indicate that binding does not fully explain the interaction between nyasol and the estrogen receptors.

### Example 6

### MCF-7 Kidney Capsule Xenografts

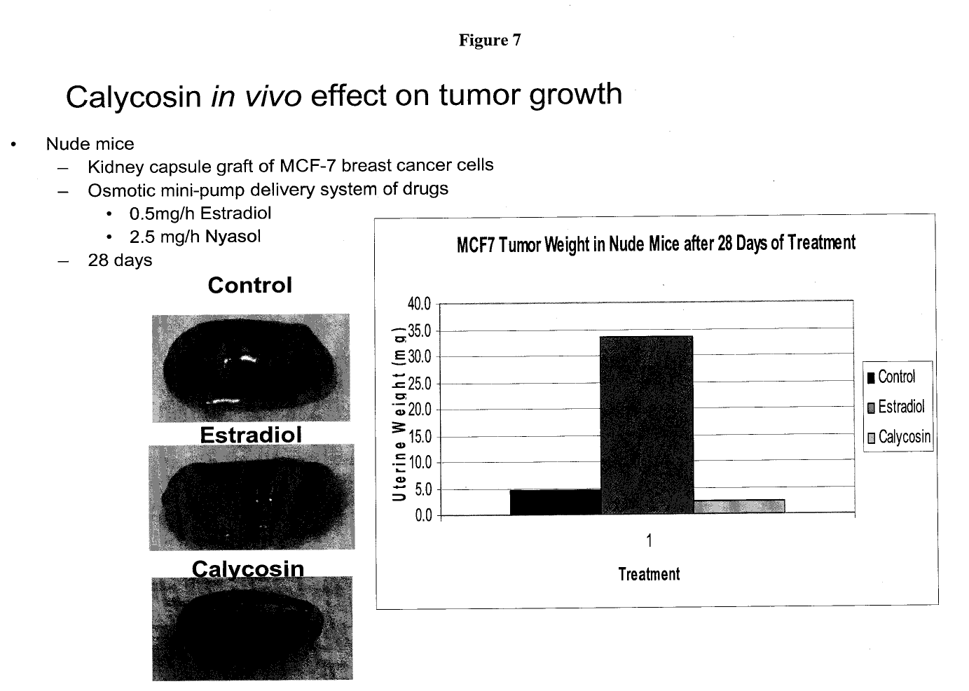

FIG. 7 shows a comparison of the effects of estradiol (E₂), Nyasol and control (carrier) on kidney capsule xenografts of MCF-7 breast cancer cells. MCF-7 xenografts were introduced into nude mouse kidneys. Mice were randomized to three treatment groups. The estradiol group received 0.5 mg/h E₂ in saline; the Nyasol group received 2.5 mg/h of Nyasol in saline; the control group received saline only. Each treatment group was treated for 28 days, after which mice were euthanized and the kidneys containing the xenografts were excised, photographed and weighed. As can be seen (FIG. 7A, 7B), estradiol agonizes tumor xenograft growth as compared to control, whereas Nyasol inhibits the growth of MCF-7 breast cancer xenografts.

### Example 7

### Nyasol's Effect on Uterine Growth in Nude Mice

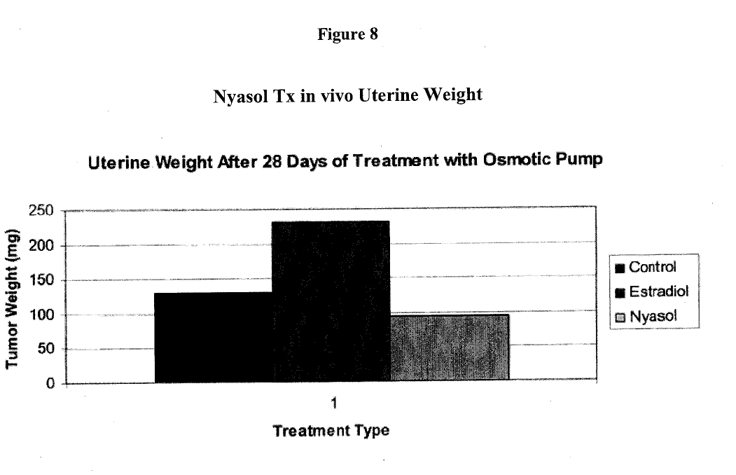

FIG. 8 shows a comparison of the effects of E₂, Nyasol and a control on in vivo uterine weight. Female nude mice were treated with either E₂, Mice were randomized to three treatment groups. The estradiol group received 0.5 mg/h E₂ in saline; the Nyasol group received 2.5 mg/h of Nyasol in saline; the control group received saline only. After 28 days, each mouse was euthanized and its uterus was removed and weighed. As can be seen, E₂ agonizes uterus growth, while Nyasol has the opposite effect, relative to control.

### Example 8

### Appendices A and B

See Appendices A and B for these experiments.

### Example 9

### Open Label, Increasing Dose, Dosing Study

In order to assess the safety and maximum tolerated dose (MTD) of a composition comprising one or more of (a), (b), (c), (d), (e), (f), (g) and (h), an open label, increasing dose study is conducted. The study drug contains one of the following compositions: I: (a) as sole active ingredient; II: (b) as sole active ingredient; III: (c) as sole active ingredient; IV: (d) as sole active ingredient; V a 1:1:1:1 mixture of (a), (b), (c), (d), (e), (f), (g) and (h).

Study Drug comprises 1 mg (week 1), 10 mg (week 2), 100 mg (week 3) or 1000 mg (week 4) of I, II, III, IV or V in a suitably sized gelatin capsules. The dose may be split between two or more gelatin capsules if necessary. Normal, healthy volunteers of age 18 to 60 are administered 1 mg per day of Study Drug for week 1, 10 mg per day of Study Drug for week 2, 100 mg per day of study drug for week 3 and 1000 mg per day of Study Drug for week 4. Subjects are monitored for appearance of any adverse events. At any time, if a subject appears to not tolerate the current dose, the attending medical staff will note such intolerance. The maximum tolerated dose will be considered the highest dose at which each of the subjects tolerates the dose, or, if no subject experiences intolerance, 1000 mg of the Study Drug per day.

### Example 10

### Double Blind Efficacy Study

In order to demonstrate efficacy of the Study Drug for the treatment of estrogenic disease states, the following double blind study is performed.

Objective: To determine optimal dose and the safety and efficacy of an ERβ-selective Chinese herbal extract (Study Drug) for treatment of hot flushes (also known as hot flashes).

Methods: A multicenter, randomized, blinded, phase II, placebo-controlled trial in 100-300 generally healthy postmenopausal women aged 40-60 years reporting at least 7 moderate to severe hot flushes per day or 50 per week. Women are randomized to 5 g (SG5) or 10 g (SG10) per day of Study Drug or identical placebo (PG) for 12 weeks. Hot flush frequency and severity are recorded in a daily diary.

Results: Participants are characterized by mean age and race. Participants receiving both Study Drug and placebo are also characterized by percent decrease (±S.D., and p value) in hot flush frequency after 12 weeks of treatment. Endometrial thickness is evaluated for each participant and each group (overall, PG, SG5, SG10). Adverse events are also evaluated for each participant and each group (overall, PG, SG5, SG10).

Conclusions: Evaluation is based upon the reduction in frequency and severity of hot flushes in healthy postmenopausal women as well as dose titration effects.

Methods

Design and Setting: This is a multi-center, randomized, blinded, placebo-controlled trial designed to determine whether the Study Drug is safe and effective in reducing the frequency and severity of hot flushes. The trial is coordinated through an independent third party (Coordinating Center) and participants are recruited at multiple clinical sites.

Participants

Eligible participants are generally healthy postmenopausal women 40 to 60 years old who reported at least 7 moderate to severe hot flushes per day or 50 per week. Women who are excluded: those with a history of breast, uterine or ovarian cancer; melanoma; venous thromboembolism; cardiovascular disease, or severe food or medicine allergies. Also excluded are women reporting active liver or gallbladder disease; abnormal uterine bleeding; pregnancy or lactation, and those with an abnormal mammogram, breast examination, Pap smear or pelvic examination suggestive of cancer. Women with endometrial thickness exceeding 5 mm measured by transvaginal ultrasound and those using medications known or suspected to affect hot flushes (estrogens, tamoxifen, raloxifene, progestins, selective serotonin reuptake inhibitors or gabapentin) are also excluded.

At screening, placebo medication and diaries to record hot flushes, bleeding and medication adherence are provided for a 1-week run-in period. Participants who correctly complete their diaries, take at least 80% of the placebo medication, and remain eligible after screening physical, radiological, and laboratory exams are randomized.

Drug safety is evaluated by a Data Safety and Monitoring Board.

Data Collection

Data are collected, cleaned and analyzed by the Coordinating Center.

Randomization

Randomization is stratified by time since last menstrual period (<24 months vs. >24 months) and by clinical site; within strata, treatment is randomly assigned in randomly permuted blocks of 3 to 6 in a 1:1:1 ratio. A research pharmacist at the Coordinating Center receives the study medication from Bionovo, Inc. (Emeryville, Calif.), applies labels with treatment identification numbers generated by the Coordinating Center statistician, and ships study medication to each clinical site. Study medication is allocated to eligible participants sequentially according to the randomization scheme.

Study Medications and Blinding: Study Drug is a filtered, dried extract of herb as described herein. Carmel coloring and food dyes approved by the US Food and Drug Administration are added to the dry powder to reach a uniform color, and flavorings and sweeteners are added to mask the taste of the herbs. Similar coloring and taste excipients are added to inert solid diluent to produce a placebo powder with the same look, taste and granularity as the active medication.

Participants receive placebo or one of the two doses of Study Drug packaged as a powder and are instructed to dissolve the contents of the packet in at least 3 ounces of non-citrus fluid and drink the beverage twice daily. All investigators, study staff, laboratory personnel and participants are blinded to study medication status.

Measurements: At baseline, participants complete questionnaires regarding demographics, medical, history, medications, quality of life, menopausal symptoms, insomnia (Insomnia Severity Index) and sexual function (Female Sexual Function Index). All participants receive a physical examination, including blood pressure and heart rate, a breast and pelvic exam, and, in women without a hysterectomy, a transvaginal ultrasound to measure endometrial double wall thickness. To evaluate safety, serum hematology, creatinine and urea nitrogen, liver function, and a urine analysis are all performed for each patient. All baseline measures are repeated after 12 weeks of treatment or at the final study visit.

Hot flush frequency and severity are recorded on a diary modeled after a diary widely used in prior studies. The 7-day diary is completed prior to randomization and during weeks 4 and 12 on study medication. For each hot flush, severity is rated as 1 (mild), 2 (moderate) or 3 (severe). A hot flush score is calculated by adding the severity rating for each hot flush and dividing by the number of hot flushes.

While on study medication, participants are contacted (by phone or in the clinic) at 2 and 8 weeks, and have a clinic visit at 4 weeks to monitor adherence and adverse events. Medication packets are counted to assess adherence; and adverse events are recorded.

Four weeks after discontinuing study medication, each participant is contacted by phone to ascertain information on adverse events. Self-reported adverse events are classified using the Medical Dictionary for Regulatory Activities (MedDRA) system.

Diagnostic endometrial biopsies are performed during the study if a participant reports vaginal spotting or bleeding, or if the final endometrial wall thickness measured by transvaginal sonography is over 5 mm or has increased 2 mm or more from baseline. Two blinded pathologists evaluate biopsy specimens, if any, independently. If the pathologists disagree regarding histology, another third blinded pathologist reviews the slide and makes the final diagnosis.

Statistical Analysis: A sample of 180 participants is estimated to provide 80% power to detect a between-group difference of 20 percentage points in the percent change in hot flush frequency from baseline to 12 weeks.

All analyses are by intention to treat, according to randomized assignment, without regard to adherence and without imputing or carrying forward missing values. No adjustment is made for multiple testing. Baseline characteristics of the participants are compared using linear or logistic regression or proportional odds models controlling for clinical center and years since menopause.

Primary analyses compare changes from baseline to 4 and 12 weeks in frequency of hot flushes and hot flush score between each of the Study Drug groups (SG5 and SG10) and placebo (PG). Because the outcomes are right-skewed, repeated-measures log-link Poisson generalized linear models with terms for time (4 or 12 weeks vs. baseline), treatment, and a time-by-treatment interaction, as well as clinical center and years since menopause are used. Primary analyses of secondary outcomes (quality of life, sexual function and insomnia scores) use analogous methods.

In secondary analyses, ANCOVA is used, controlling for site and time since menopause to compare rank transformed percent change in number of hot flushes between the treated and placebo groups. Logistic regression models adjusted for clinical site and years since menopause are used to compare the proportions in each treatment group with a reduction in frequency of hot flushes of 50% or greater from baseline to 12 weeks.

The frequency of adverse events that occurs in more than 2% of any of the treatment groups is compared between treatment groups using chi-square and exact methods when appropriate, stratified by clinical center and years since menopause.

In pre-specified exploratory analyses, interaction terms are used to determine differences in the treatment effect (percent change in hot flushes at 12 weeks) in subgroups including age (45-50; 50-55; 55-60 years) ethnicity (white, other), years since menopause (less than 2 years; 2 years or more), bilateral oophorectomy (yes; no), history of estrogen use (yes; no), smoking (current; former or never), current alcohol use (yes, no), body mass index (tertiles), baseline serum estradiol level (5 pg/ml or less; greater than 5 pg/ml), and baseline frequency of hot flushes (tertiles).

Results

Results include number of eligible women who are randomized; number of women in each group (PG, SG5, SG10); number of participants who complete the study overall and in each group and strata; number of participants overall and in each group who took all the assigned medication; number of white and non-white participants overall and in each group; baseline median and mean daily frequency of hot flushes (±S.D., p); median and mean daily hot flush score (±S.D., p); median and mean change in hot flush frequency (±S.D., p) and median and mean hot flush score (±S.D., p) at each evaluation interval.

The effects of treatment with Study Drug on measures of quality of life, sleep quality and sexual function as compared to placebo are also evaluated.

The number of participants receiving transvaginal ultrasound at baseline and the end of the study is also noted. The number of participants receiving endometrial ultrasound at the end of the trial is also noted. Mean endometrial thickness (+S.D.) at baseline and at 12 weeks is measured. Where deemed necessary, endometrial biopsy is also performed. The number of participants reporting vaginal bleeding or spotting is also noted; and endometrial biopsy is in as many of these participants as grant consent. The biopsies are evaluated for evidence of endometrial hyperplasia and cancer.

Any serious adverse events during the trial are also noted.

Discussion

It is considered that treatment with the Study Drug will decrease the frequency and severity of hot flushes in healthy postmenopausal women with moderate to severe symptoms. The results of this study may be used to advance the Study Drug on to further clinical trials, in which the same or higher doses of Study Drug may be tested.

It is also considered that, as the Study Drug is a selective ERβ agonist, adverse events associated with estrogen replacement therapy, such as uterine hyperplasia and cancer, should not be observed for the Study Drug.

While estradiol is an effective treatment for menopausal hot flushes, the currently approved selective estrogen receptor modulators (SERMS) tamoxifen and raloxifene increase the incidence of menopausal hot flashes. Since neither estradiol nor the SERMs are estrogen receptor subtype selective, it is unclear which estrogen receptor, ERα or ERβ mediates these effects. It has been shown that activation of ERα by estrogen in human breast cancer cells results in proliferation and tumor formation, while activation of ERβ results in growth inhibition and no tumor formation. This study is designed to provide data to demonstrate that hot flushes may be relieved by the Study Drug. This study is further designed to provide preliminary data regarding adverse events that may be associated with the Study Drug.

Conclusion: Treatment with the Study Drug is expected to reduce the frequency and severity of hot flushes in healthy postmenopausal women; and the higher dose of the Study Drug is expected to be more effective than the lower dose. This study is furthermore expected to provide further confirmation that the ERβ pathway may play a role in the treatment of hot flushes.

Although the invention has been illustrated with reference to certain embodiments and examples, the person having skill in the art will recognize that other embodiments are envisioned within the scope of the present invention.

### CONCLUSION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1-16. (canceled)

17. A method of eliciting an estrogenic effect, comprising administering to a subject an estrogenically effective amount of one comprising an amount of at least one isolated and purified member of the group consisting of compounds (a), (b), (c), (d), (e), (f), (g) and (h), wherein the amount is sufficient to modulate estrogen receptor beta (ERβ) in a multicellular organism:

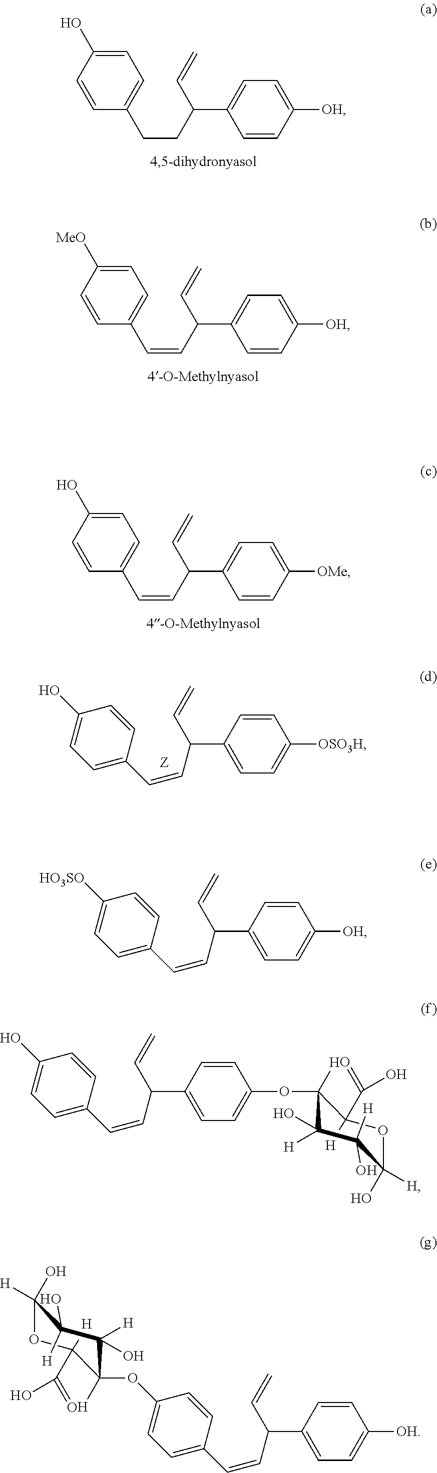

18. The method of claim 17, wherein the composition comprises two or more of (a), (b), (c), (d), (e), (f), (g) and (h).

19. The method of claim 17, wherein the composition comprises three or more of (a), (b), (c), (d), (e), (f), (g) and (h); or each of (a), (b), (c), (d), (e), (f), (g) and (h).

20. (canceled)

21. The method of claim 17, wherein the estrogenic effect is at least one effect selected from the group consisting of: treating or preventing at least one climacteric symptom; treating or preventing osteoporosis; treating or preventing uterine cancer; and treating or preventing cardiovascular disease.

22. The method of claim 21, wherein the estrogenic effect includes treating or preventing at least one climacteric symptom selected from the group consisting of treating or preventing hot flashes, insomnia, vaginal dryness, decreased libido, urinary incontinence and depression.

23. The method of claim 21, wherein the estrogenic effect includes treating or preventing osteoporosis.

24. The method of claim 21, wherein the estrogenic effect includes treating or preventing hot flashes.

25. The method of claim 21, wherein the estrogenic effect includes treating or preventing uterine cancer.

26. The method of claim 17, wherein the estrogenic effect does not include increasing the risk of mammary hyperplasia, mammary tumor, uterine hyperplasia, uterine tumor, cervical hyperplasia, cervical tumor, ovarian hyperplasia, ovarian tumor, fallopian tube hyperplasia, fallopian tube tumor.

27. The method of claim 17, wherein the estrogenic effect includes decreasing the risk of mammary hyperplasia, mammary tumor, uterine hyperplasia, uterine tumor, cervical hyperplasia, cervical tumor, ovarian hyperplasia, ovarian tumor, fallopian tube hyperplasia, fallopian tube tumor.

28. A method of activating a gene under control of an estrogen response element, comprising administering to a cell having an estrogen response element operatively linked to the gene and an estrogen receptor an amount of a composition of claim 1 sufficient to activate said gene.

29-41. (canceled)

42. A method of repressing expression of a TNF RE-controlled gene, comprising administering to a cell comprising a gene under control of a TNF response element and an estrogen receptor an amount of a composition of claim 1 effective to repress said TNF RE-controlled gene.

43-56. (canceled)

57. A method of preparing nyasol, comprising:
(a) protecting the hydroxy group of 4-iodophenol with MOMCl to form a protected intermediate: 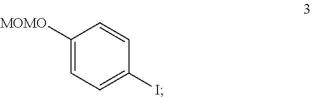
(b) contacting 3 with trimethylsilylacetylene in the presence of bis[triphenylphosphine]palladium dichloride and CuI in diethylamine to form: 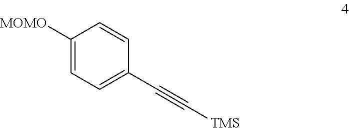
(c) reacting 4-hydroxybenzadehyde with MOMCl to form: 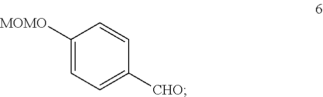
(d) reacting 6 with ethynlymagneciumbromide to form: 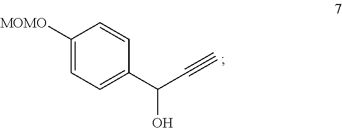
(e) reacting 4 and 7 to form 8: 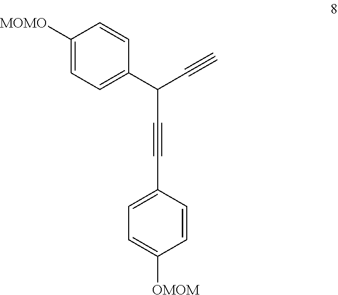
(f) reducing the triple bonds in 8 to form 9: 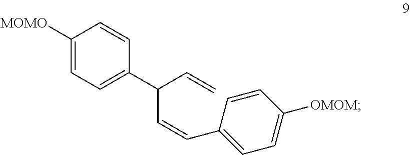
and
(g) removing the MOM protective groups to form 1 (nyasol): [chemical structure]
